(19) United States
(12) Patent Application Publication
(10) Pub. No.: US 2025/0057477 A1
(43) Pub. Date: Feb. 20, 2025

(54) INTRAORAL SYSTEMS AND METHODS INCLUDING SENSOR ASSEMBLIES FOR DETECTING AIR FLOW

(71) Applicant: Align Technology, Inc., San Jose, CA (US)

(72) Inventors: Avi KOPELMAN, Palo Alto, CA (US); Srinivas KAZA, Mountain View, CA (US)

(21) Appl. No.: 18/939,465

(22) Filed: Nov. 6, 2024

Related U.S. Application Data
(63) Continuation of application No. 16/152,281, filed on Oct. 4, 2018, now Pat. No. 12,171,575.
(60) Provisional application No. 62/568,212, filed on Oct. 4, 2017.

(51) Int. Cl. A61B 5/00; A61B 5/145; A61C 7/08; A61C 19/04

(52) U.S. Cl. CPC A61B 5/682; A61B 5/0088; A61B 5/14503; A61B 5/14514; A61B 5/4552; A61B 5/685; A61B 5/6867; A61B 5/14507; A61B 5/4845; A61B 5/688; A61B 2562/046; A61B 2562/066; A61C 7/08; A61C 19/04

(57) ABSTRACT

Intraoral systems and methods with sensor assemblies for detecting air flow. The systems may include a removable polymeric shell appliance including cavities for receiving a patient's teeth. A sensor assembly may be coupled to the polymeric shell appliance. The sensor assembly may include a power source, a sensor that is configured to detect air flow during inhalation and/or exhalation of the patient when the patient is wearing the polymeric shell appliance, a memory for storing air flow data based on the detected air flow, and wireless circuity for transmitting information associated with the air flow data to an external device.

### CROSS REFERENCE TO RELATED APPLICATIONS

This patent application is a continuation of U.S. patent application Ser. No. 16/152,281, filed on Oct. 4, 2018, titled “INTRAORAL SYSTEMS AND METHODS FOR SAMPLING SOFT-TISSUE,” now U.S. Patent Application Publication No. 2019/0099129, which claims priority to U.S. Provisional Patent Application No. 62/568,212, filed on Oct. 4, 2017, titled “INTRAORAL APPLIANCES FOR SAMPLING SOFT-TISSUE,” each of which is herein incorporated by reference in its entirety.

This application may be related to U.S. patent application Ser. No. 16/019,037, filed on Jun. 26, 2018 (“BIOSENSOR PERFORMANCE INDICATOR FOR INTRAORAL APPLIANCES”), which claimed priority to U.S. provisional patent no. 62/525,082, filed Jun. 26, 2017 (“BIOSENSOR PERFORMANCE INDICATOR FOR INTRAORAL APPLIANCES”), each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are gingival sensors for sensing one or more analytes from the gingiva while being stably held in position against the teeth.

### BACKGROUND

Blood and oral fluids that are obtained from the tissues surrounding the teeth may be used for diagnostic or therapeutic purposes. Typically samples of blood or other fluids are extracted by an external sampler (such as a needle) and sampled and/or analyzed external systems that examine the fluid to identify analytes of interests. Such techniques may make it difficult to provide longer, longitudinal analyses or continuous monitoring.

Further, such methods are typically not suitable for infants, disabled patients, patient suffering from needles fear and patients that need frequent body fluids sampling, mass monitoring and ordinary patients asking to spare the inconvenience that may be involved.

### SUMMARY OF THE DISCLOSURE

Described herein are apparatuses, including systems and devices, configured to secure one or more sensors against a soft tissue while remaining secured to a hard tissue. In particular, described herein are sensors and sensor holders that are configured to couple to a hard tissue, such as the teeth, for accessing a soft tissues, such as the gingiva. In general, these apparatuses, and methods of using them, may include an intraoral appliance, including but not limited to an aligner, that is configured to secure to a patient's teeth and hold one or more sensors, including in particular soft tissue sensors.

For example, described herein are devices including a hard tissue anchoring portion having a tooth-holding channel or region that is configured to fit over all or portion of a patient's dental arch, and a one or more sensors attached (removably or permanently attached) that extends from the hard tissue anchoring portion towards the soft tissue, such as the patient's gingiva. The sensor may include one or more microneedles for sampling from the soft tissue. For example, any of the apparatuses described herein may include a sensor having one or more microneedles for accessing to the patient's soft tissue.

The hard tissue anchoring portion generally removably attaches to the patient's teeth. The hard tissue anchoring portion may be configured as a dental appliance that can be applied and removed over the patient's teeth. The dental appliance may be an intraoral appliance that is configured to set securely onto the patient's dental arch. The dental appliance may be secured by friction fit, onto the teeth. The dental appliance may be secured to all, or substantially all, of the patient's teeth in the upper and/or lower jaw, or it may be secured to just a subset of the patient's teeth in the upper or lower jaw.

For example, described herein are intraoral, soft-tissue sensor systems that include: an intraoral appliance shaped to receive a patient's teeth within a cavity and to secure the intraoral appliance against the patient's teeth; a soft tissue sensor mounted to the intraoral appliance, wherein the soft tissue sensor comprises one or more microneedles projecting from the intraoral appliance that are configured to penetrate soft tissue adjacent to the patient's teeth when worn, wherein the soft tissue sensor is configured to detect at least one analyte through the one or more microneedles; and a bias coupled to the soft tissue sensor configured to apply a biasing force to the soft tissue sensor. The bias may be activated, so that the sensor and/or the microneedle is held further away from the gingiva when initially worn, and once the intraoral appliance is worn on the teeth, the bias may be triggered (activated) to extend the microneedle (which may generally include both tissue penetrating and non-penetrating microneedles) towards, against, and/or into the soft tissue such as the gingiva and/or palate.

The soft tissue sensor may be removably mounted to the intraoral appliance, or permanently mounted to the intraoral appliance. A soft tissue sensor may include a fluid sensor that is configured to sense a property (electrical, chemical, mechanical, etc.) from the soft tissue such as the gingiva, and/or saliva, interstitial fluid, lymph, or blood associated with the gingiva. For example, the soft tissue sensor may be a sensor that penetrates into the soft tissue, such as the gingiva, in order to detect and/or measure a property from within the tissue. Alternatively or additionally, the soft tissue sensor may be configured to sample a fluid from within the gingiva by penetrating the soft tissue (e.g., with one or more hollow or solid microneedles) and allow the fluid to make contact with the needle or an inner lumen of the needle. A soft tissue sensor may be an electrical sensor that is configured to contact the soft tissue (without penetrating or with penetrating) and measure one or more properties.

The soft tissue sensor may generally be positioned on the intraoral appliance so as to not interfere with the patient's bite when worn. For example, the sensor may be held on an outer buccal or lingual side of the appliance, but may avoid the occlusal surface. The soft tissue sensor may also be shaped so that when engaged with the appliance, it does not protrude or provide any abrupt and/or rough surfaces. For example, one or more outward-facing surfaces or sides of the soft tissue sensor may be smooth and/or curved so that the tongue does not scrape against it.

The soft tissue sensor, and particularly any portion mounted or configured to be mounted on the buccal side of the intraoral appliance, may be configured so as to blend in with the appliance and/or the patient's teeth. For example, the soft tissue sensor maybe formed of a translucent or transparent material. Alternatively, the soft tissue sensor may be colored to match the teeth and/or the intraoral appliance.

The soft tissue sensor may be configured to passively sense analyte or to actively sense analyte. For example, the soft tissue sensor include an optical sensor that illuminates a portion of the tissue with a specific wavelength and receives reflected and/or transmitted light passing through the soft tissue, blood, lymph, interstitial fluid, and/or saliva. The soft tissue sensor may be configured to extract material from the soft tissue (e.g., blood, saliva, interstitial fluid, lymph, etc.). The soft tissue sensor may comprise a chemical, immunohistochemical, enzymatic, or other sensor. Optical sensors may include near- and mid-IR sensors, florescence sensors (e.g., FRET), optical coherence tomography sensors, spectrographic sensors, etc. An optical sensor may be coupled with the microneedle and/or may sample material from the microneedle. An optical fiber may be held with or within the microneedle.

Any of the soft tissue sensors described herein may be configured to deliver material to the soft tissue, e.g., through the microneedle.

In general, the microneedle may be any appropriate needle, hollow or solid, that is 1 millimeter or less in diameter (e.g., 0.9 mm or less, 0.8 mm or less, 0.7 mm or less, 0.6 mm or less, 0.5 mm or less, 0.4 mm or less, 0.3 mm or less, 0.2 mm or less, 0.1 mm or less, etc.). The microneedle may extend from a base or substrate by any appropriate length (e.g., between 0.5 mm and 5 cm, e.g., between 0.5 cm and 4 cm, between 0.5 mm and 3 cm, between 0.5 mm and 1 cm, between 0.5 mm and 9 mm, between 0.5 mm and 8 mm, between 0.5 mm and 7 mm, between 0.5 mm and 6 mm, between 0.5 mm and 5 mm, etc.). The microneedle may be fabricated from any appropriate material, including metal, silicon (or silicon dioxide), polymer, etc., including combinations of these.

In general, any of the apparatuses described herein may include a bias (e.g., spring, elastic, etc.) that is configured to hold or drive the sensor or a sensing portion of the sensor (e.g., microneedle) against the soft tissue or to otherwise maintain contact and/or penetration with the soft tissue. In some variations the apparatus includes a spring bias. Any spring bias may be included (e.g., coil spring, flat spring, serpentine spring, leaf spring, v-spring, etc., gas spring, etc.). The bias may be configured to apply a force, and particularly a continuous force, on the soft tissue sensor to hold the sensor against the patient's soft tissue. The spring may be configured to limit the maximum amount of force applied (e.g., to less than 1 N, less than 0.5 N, less than 0.1 N, etc.). The bias may be part of the sensor (e.g., on an outer surface of the sensor) or part of the intraoral appliance holding the sensor.

Any of the intraoral appliances described herein may be configured to couple to the teeth, as mentioned above. In addition, the intraoral appliance may be configured to couple to the palate (soft and/or hard palate). For example, any of the intraoral appliances described herein may include a palatal region covering all or a portion of the palate. Further, any of these apparatuses may include one or more temporary anchorage devices.

Any of these intraoral appliances may be configured to mount to the hard tissue, including the teeth. For example, any of these apparatuses may include one or more attachment sites on the intraoral appliance configured to couple with the one or more temporary anchorage devices. Attachments may be affixed to the outside (e.g., buccal and/or lingual sides) of a tooth or teeth, e.g., by bonding. The attachment may couple to an attachment receiver on the intraoral appliance. Alternatively or additionally, the intraoral appliance may be configured to screw into a tooth, including onto a prosthetic tooth.

Any appropriate soft tissue sensor may be used, and may be configured to detect one or more analytes. For example, the soft tissue sensor may be configured to detect one or more of: electrolytes (e.g., fluoride, sodium, etc.), pH, glucose level, hydration, white blood cell count, lactate level, an inflammatory marker, a bacterial marker, air flow during inhaling and/or exhalation; chemical content of air inhaled; chemical content of air exhaled; bad breath, alcohol level.

The intraoral appliance provide a stable base or frame against which the sensor(s) may be held to the soft tissue, allowing sable sensing from the soft tissue. The intraoral appliance may also permit one or more sensors to be attached at more than one location, allowing rotation of the position of the sensor within the patient's mouth, preventing or reducing irritation. For example, the apparatuses described herein may allow a sensor to be attached to the intraoral appliance at a first position that allow sampling from a first region of the patient's mouth (anterior, etc.). The same sensor or a different sensor, including a different sensor of the same type (e.g., also including a microneedle) may then be moved, in the same intraoral appliance or another intraoral appliance, relative to the first positon, to a second position, allowing sampling of a separate region of the patient's mouth for a second time period (e.g., until removal of the intraoral appliance, such as one hour, 2 hours, 3 hours, 4 hours, 5 hours, 12 hours, 1 day, 2 days, 3 days, etc.).

Also described herein are methods of using any of the apparatuses described herein, including in particular, described herein are methods of monitoring (including methods of continuously monitoring) or detecting an analyte from a patient's soft tissue. For example, a method of detecting an analyte from a patient's soft tissue may include: placing an intraoral appliance onto the patient's teeth so that the patient's teeth are secured within a cavity of the intraoral appliance; biasing a soft tissue sensor mounted to the intraoral appliance against the soft tissue, wherein the soft tissue sensor comprises one or more microneedles projecting from the intraoral appliance, further wherein biasing comprises applying a biasing force to the soft tissue sensor to penetrate a soft tissue adjacent to the patient's teeth; and sensing an analyte through the one or more microneedles.

As mentioned, biasing may comprise applying a continuous force. The biasing force may be variable or constant. For example, biasing may comprise applying a continuous force from a spring. The method of sensing the analyte may comprise detecting one or more of: air flow during inhaling and/or exhalation; chemical content of air inhaled; chemical content of air exhaled; bad breath, alcohol level, glucose level, an inflammatory marker, or a bacterial marker.

Other apparatuses described herein may include intraoral, soft-tissue sensor systems in which the sensor (e.g., a sensor with one or more microneedles) may removably attached from the hard-tissue secured portion, e.g., an intraoral appliance. For example an intraoral soft-tissue sensing system may include: an intraoral appliance shaped to receive a patient's teeth within a cavity and to secure the intraoral appliance against the patient's teeth; and a soft tissue sensor detachably mounted to the intraoral appliance, wherein the soft tissue sensor comprises one or more microneedles projecting from the intraoral appliance that are configured to penetrate soft tissue adjacent to the patient's teeth when worn, wherein the soft tissue sensor is configured to detect at least one analyte from the patient's soft tissue through the one or more microneedles.

Any of these apparatuses may be configured to lock or otherwise secure the soft tissue sensor to the intraoral appliance. The intraoral appliance may include a snap, claps, strap, button, or any other fastener that may be used to secure the sensor in a fixed position in/on the intraoral appliance. The lock may be releasable or not releasable (e.g., single-use, etc.).

Any of the variations described above, may be incorporated into the apparatuses having a removable sensor.

Also described herein are intraoral, soft-tissue sensor systems that are configured to receive a sensor, but which do not include the sensor. For example, the sensor may be provided by a third party that uses the apparatus described herein to hold the sensor. For example, any of the apparatuses described herein may be configured to include a mount, which may have a standard or typical size, for receiving a sensor, so as to hold the sensor in stable position (relative to the hard tissue, e.g., teeth) for sensing from a soft tissue. The holder may be configured to releasably hold the sensor. For example, described herein are intraoral, soft-tissue systems (configured a stable sensor mounts) including: an intraoral appliance shaped to receive a patient's teeth within a cavity and to secure the intraoral appliance against the patient's teeth; and a soft tissue sensor mount on an outer surface of the intraoral appliance, wherein the soft tissue sensor mount is configured to detachably mount a soft tissue sensor to the intraoral appliance. Optionally, any of these apparatuses may include a soft tissue sensor configured to mount to the soft tissue sensor mount so that one or more microneedles project from the intraoral appliance to penetrate soft tissue adjacent to the patient's teeth when the intraoral appliance is worn by the patient. The soft tissue sensor may be configured to detect at least one analyte from the patient's soft tissue through the one or more microneedles.

For example, an intraoral, soft-tissue sensor system customized to fit a patient's dentition may include: a plurality of intraoral appliances, wherein each intraoral appliance is shaped to receive the patient's teeth within a cavity and to secure the intraoral appliance against the patient's teeth, further wherein each intraoral appliance is divided up into a plurality of regions; a soft tissue sensor mounted on each of the intraoral appliances, wherein the soft tissue sensor mounted on each of the intraoral appliances comprises one or more microneedles projecting from the intraoral appliance to which it is mounted, wherein the one or more microneedles are configured to penetrate soft tissue adjacent to the patient's teeth when worn to detect at least one analyte through the one or more microneedles; and wherein the soft tissue sensor mounted on each of the intraoral appliances is mounted to a different regions of each of the intraoral appliances compared to the other intraoral appliances in the plurality of intraoral appliances.

As mentioned above, any of the intraoral appliances in the plurality of intraoral appliances may include a bias coupled to the soft tissue sensor mounted on the intraoral appliance, wherein the bias is configured to apply a biasing force to the soft tissue sensor to secure the soft tissue sensor against and/or in the soft tissue when worn.

The different regions of the plurality of regions may be separated from each other by any appropriate distance. For example, the different regions of the plurality of regions may be separated from each other by a separation of greater than 0.5 mm (e.g., greater than about: 0.6 mm, 0.7 mm, 0.8 mm, 0.9 mm, 1 mm, 1.1 mm, 1.2 mm, 1.3 mm, 1.4 mm, 1.5 mm, 2 mm, etc.).

The plurality of intraoral appliances may be arranged in an ordered sequence such that, after the first intraoral appliance in the sequence, the region to which the soft tissue sensor is mounted in each subsequent intraoral appliance in the ordered sequence are not adjacent to the region to which the soft tissue sensor is mounted in the proceeding intraoral appliance in the ordered series.

In any of these intraoral appliances, the cavity may be configured to hold the patient's upper or lower arch. As mentioned above, each intraoral appliance of the plurality of intraoral appliances may include a palatal region configured to be positioned adjacent to the patient's palate when worn (either touching or not touching, e.g., separately by a distance of greater than about 0.1 mm, 0.2 mm, 0.3 mm, 0.4 mm 0.5 mm, 0.6 mm, 0.7 mm, 0.8 mm, 0.9 mm, 1 mm, etc.).

Any of the intraoral appliance described herein (including those of a series, e.g., plurality of intraoral appliances) may be configured to receive the patient's molar tooth or teeth, and/or the patient's incisor tooth or teeth, and/or the patient's canine tooth or teeth.

Also described herein are systems including a plurality of intraoral appliances in which each of the appliances includes a different sensor mount location. For example, an intraoral, soft-tissue sensor system customized to fit a patient's dentition may include: a plurality of intraoral appliances, wherein each intraoral appliance is shaped to receive the patient's teeth within a cavity and to secure the intraoral appliance against the patient's teeth, further wherein each intraoral appliance is divided up into a plurality of regions extending along the length of the intraoral appliance; a soft tissue sensor mount on an outer surface of each of the intraoral appliances of the plurality of intraoral appliances, wherein each soft tissue sensor mount is configured to detachably secure a soft tissue sensor to the intraoral appliance, further wherein the soft tissue sensor mounts on each of the intraoral appliances are located on different regions of each of the intraoral appliances compared to the locations of the soft tissue sensor mounts on each of the other intraoral appliances in the plurality of intraoral appliances.

For example, described herein are system wherein the soft tissue sensor mounts on each of the different intraoral appliance mounting locations and each intraoral appliances comprises a bias configured to couple to a soft tissue sensor and to apply a biasing force to the soft tissue sensor.

Any of these apparatuses (including these systems) may include a soft tissue sensor configured to mount to the soft tissue sensor mounts of each of the plurality of intraoral appliances so that one or more microneedles of the soft tissue sensor project to penetrate soft tissue adjacent to the patient's teeth when the intraoral appliance is worn by the patient, further wherein the soft tissue sensor is configured to detect at least one analyte from the patient's soft tissue through the one or more microneedles.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a is an example of an apparatus configured as an intraoral, soft-tissue sensor system, including an intraoral appliance shaped to receive a patient's teeth and to secure the intraoral appliance against the patient's teeth, and a soft tissue sensor mounted to the intraoral appliance, wherein the soft tissue sensor comprises one or more microneedles projecting from the intraoral appliance that are configured to penetrate soft tissue adjacent to the patient's teeth when the apparatus is worn. In FIG. 1A, the sensor is held in a sensor mount at a first location relative to the intraoral appliance, and multiple microneedles extend down from the sensor.

FIG. 1B is another example of an apparatus, similar to that shown in FIG. 1A, in which the sensor is held in a mount on the intraoral appliance in a second location, different from the first location shown for the mount and sensor in FIG. 1A.

FIGS. 2A-2D illustrate examples of different variations of sensor mounts that may be included as part of an intraoral appliance of the apparatuses described herein. In FIG. 2A the sensor mount is shown with a sensor inside of the mount, surrounded on four (of six) sides of the mount (open on the face and bottom). In FIG. 2B, the sensor mount includes a bias driving the sensor held within the mount down (e.g., towards the gingival tissue). In FIG. 2C the sensor mount is enclosed on all but one side (e.g., the bottom) with the sensor secured within it. This variation may also include a bias. In this example, the sensor includes a single microneedle. FIG. 2D shows another example, similar to that shown in FIG. 2B in which the sensor mount includes a bias driving a single probe, configured as a non-penetrating probe, down onto the tissue.

FIGS. 3A-3C illustrate an example of a sensor mount on an intraoral appliance having a bias that can be activated (e.g., by manually pushing a button, upon wearing the intraoral appliance on the teeth, or by a remote signal, etc.) to extend, and in some cases retract, the sensor and/or the microneedles from the intraoral appliance. In FIG. 3A the sensor and microneedles are retracted. In FIG. 3B the bias has been activated driving the microneedles down, e.g., into the soft tissue. Optionally, the sensor and/or sensor mount may be configured so that the bias may be deactivated or retracted, withdrawing the needles, as shown in FIG. 3C.

FIG. 4 illustrates the application of an apparatus such as that shown in FIGS. 1A or 1B onto the patient's teeth, so that when secured onto the teeth, the sensor and microneedle may be in contact with the gingiva.

FIGS. 5A-5C illustrate a series of apparatuses such as those shown in FIGS. 1A and 1B, in which the sensors are arranged in different locations. The devices shown in FIGS. 5A-5C may be worn sequential (e.g., the device of FIG. 5A, then FIG. 5B, then FIG. 5C may be worn sequentially).

FIG. 6 is a schematic illustrating one example of a sensor (including a microneedle, shown as a hollow microneedle) that may be used to sense one or more analytes. The soft tissue sensor includes a bio-receptor portion for recognizing the analyte, and a signal transduction portion for transducing the signal sensed, and a detector for indicating that a signal (and optionally the intensity of the signal) representing the analyte has been detected.

FIG. 7A schematically illustrates one example of an apparatus including a monitoring device (e.g., electronics) that may include a biosensor or may communicate with a biosensor coupled to the oral cavity (e.g., teeth, gingiva, etc.) and one or more microneedle(s).

FIG. 7B schematically illustrates another example of an apparatus including a biosensor that may include any of the microneedles described herein.

FIG. 8A illustrates a schematic of a biosensor apparatus (e.g., which may be part of a monitoring device) with an activation mechanism that may include any of the microneedle(s) described herein.

FIG. 8B illustrate schematically an example of a monitoring device 800 with an activation mechanism that may include any of the microneedle(s) described herein.

FIG. 8C is an example of an apparatus with an activation mechanism similar to that shown in FIG. 8A. In this example, the apparatus includes one or more microneedles, and one or more bio-receptors formed on a silicon nanowire atop a substrate. Nanoparticles and/or nanowires may be used as part of the bio-receptor and may be included at least partially in or in fluid communication with the one or more microneedles.

FIG. 9 is a cross-section through an apparatus similar to that shown in FIGS. 1A-1B (e.g., a removable orthodontic device, also referred to as an orthodontic appliance) including an integrated monitoring device and one or more microneedles.

FIG. 10 illustrates an example of a method of operating a removable orthodontic device (e.g., appliance) including a biosensor.

### DETAILED DESCRIPTION

Oral sensors, and particularly soft-tissue sensors, may be used as part of an apparatus (e.g., system, device, etc.) to detect and/or monitor one or more factors relevant to a patient's health. When using a soft-tissue sensor to monitor patient health it may be helpful to hold the sensor, and particularly a microneedle associated with the soft-tissue sensor in stable contact with the soft tissue. Described herein are intraoral, soft-tissue sensor systems that may be used to hold a soft tissue sensor in contact with the gingiva.

In general, the soft tissue sensor systems described herein may include an intraoral appliance shaped to receive a patient's teeth within a cavity and to secure the intraoral appliance against the patient's teeth. The intraoral appliance may be configured to fit snugly over all or a portion of a patient's teeth in the patient's upper or lower arch. The cavity in the intraoral appliance may be formed as a negative of the patient's dentition in the patient's upper or lower arch. The intraoral appliance may be formed of any appropriate material (including polymeric material, and clear or transparent materials), and may be formed in any appropriate manner. For example, the intraoral appliance may be fabricated by a lamination technique (e.g., vacuum forming), or by an additive (e.g., 3D printing) method or casting into a mold or by milling from a block of appropriate material. A combination of multiple such fabrication techniques may also be used.

In general, the intraoral appliance is configured to hold a soft tissue sensor, and may therefore include a mount to secure the soft tissue sensor in or on the intraoral appliance. The mount may include a lock or other securement to hold the soft tissue sensor in place. Any of the soft tissue sensor systems described herein may optionally include a soft tissue sensor that is mounted to the intraoral appliance, e.g., in a soft tissue sensor mount. The soft tissue sensor may include one or more microneedles that will project from the intraoral appliance when the soft tissue sensor is coupled with the intraoral appliance. The microneedles may be configured to penetrate soft tissue adjacent to the patient's teeth when the intraoral appliance is worn on the patient's teeth. As mentioned, any appropriate soft tissue sensor may be used. In general, the soft tissue sensor may be configured to detect at least one analyte through the one or more microneedles.

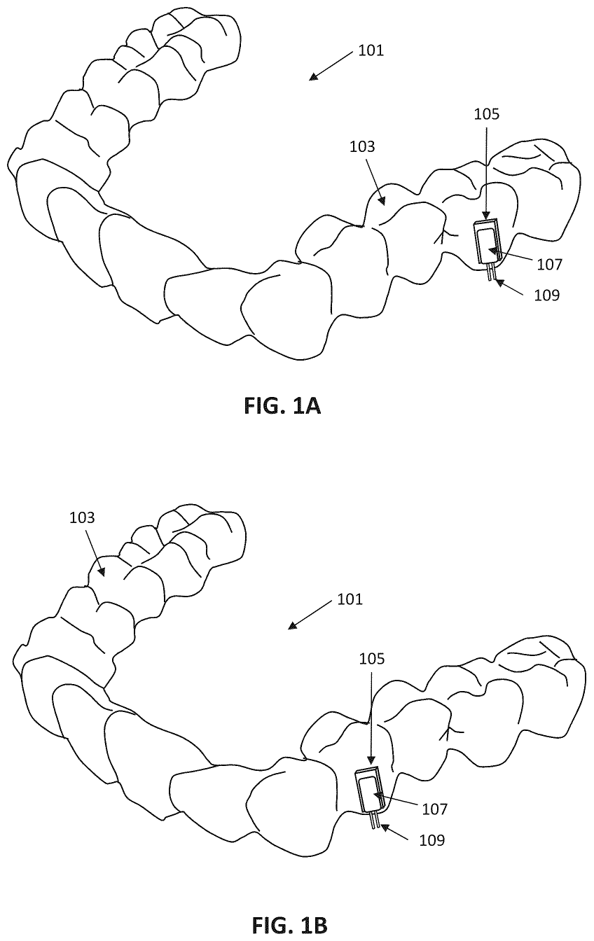

For example, FIG. 1A shows an example of a soft tissue sensor system 101. The soft tissue sensor 107 shown in this example is held in a sensor mount 105 that is formed in or connected integrally with, the intraoral appliance 103. In the example shown in FIG. 1A, the soft tissue sensor includes two microneedles that extend way from the intraoral appliance, towards the gingiva, so that the microneedles may contact the gingiva when the soft tissue sensor system is worn on the patient's teeth.

In FIG. 1A, the intraoral appliance includes the sensor mount 105 that is fixed on or in the intraoral device 103 on the left side in the second-from-the-last molar. FIG. 1B shows another example of a soft tissue sensor systems including an intraoral appliance, similar to the one shown in FIG. 1A, only positioned at a difference location. In FIG. 1B, the soft tissue sensor mount 105′ is attached to the intraoral appliance 101′ on the first (most anterior) molar on the buccal side of the teeth.

Note that in any of the soft tissue sensor systems described herein, the intraoral appliance may be formed or customized to the patient who will be wearing the apparatus.

In addition, any of the soft tissue sensor systems described herein may include a bias (e.g., coupled to the soft tissue sensor and/or a portion of the soft tissue sensor that is configured to apply a biasing force to the soft tissue sensor.

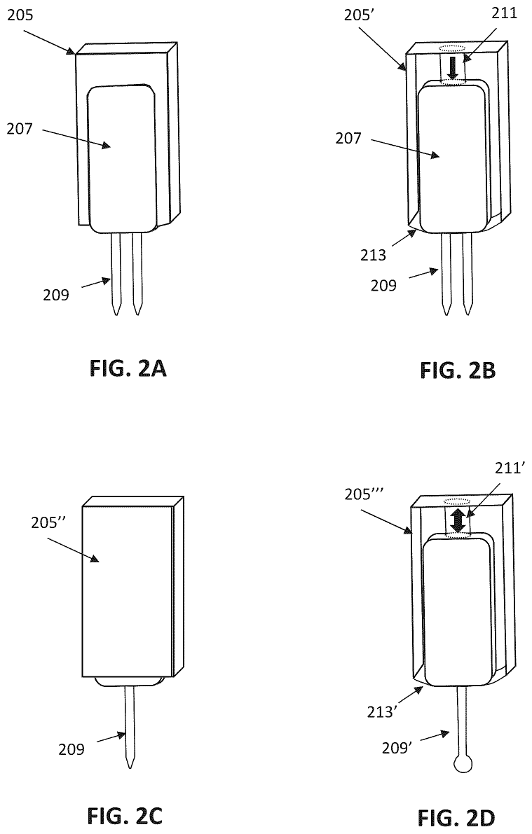

FIGS. 2A-2D illustrate examples of different soft tissue sensor mounts, each shown holding a soft tissue sensor with one (e.g., FIGS. 2C and 2D) or more, e.g., two, microneedles in communication with the sensor. The sensor may also include control logic (e.g., hardware, software firmware, including a microcontroller to coordinate the collection and distribution of data, one or more processors for processing the data, a memory for storing the data, wireless communication circuity as well as wireless circuitry for transmitting/receiving information.

In FIG. 2A, an example of a sensor holder 205 is shown with the soft tissue sensor 207 held (shown as removably held In FIG. 2A) in the sensor holder. The sensor holder may include a cavity or opening that is sized to snugly fit and hold a soft tissue sensor 207 in a fixed position, even with movements of the jaw. The sensor holder opening may be configured to hold a standard-sized sensor, or it may be custom-made to fit a specific soft tissue sensor. In FIG. 2A, the sensor is roughly rectangular with two microneedles 209 extending from the base of the sensor and out of the holder so that they may engage with the target, e.g., the gingiva. In FIG. 2A, the cavity of the sensor mount 205 may be made of a material that is slightly compressible so that the opening of the sensor mount into which the sensor is pushed, to engage the sensor in the sensor mount, may be slightly smaller than the sensor. The sensor may deflect the perimeter of the opening slightly to fit into the cavity of the sensor mount, which may then return to the original size, retaining the sensor within the cavity, which may be approximately the same size, or slightly larger, than the sensor body.

FIG. 2B shows a sensor mount 205′ that includes a bias 211 that may drive the microneedles 209 and/or the sensor 207 down, towards the gingiva, to make and/or maintain contact with the soft tissue. In FIG. 2B, the outer surface of the mount is not shown; however a cover may be used (see, e.g., FIG. 2C), or it may include an opening, similar to that shown in FIG. 2A. In FIG. 2B, the bottom of the mount 213 may be deformable and/or made form an clastic material, as shown. This may allow the bias element to deflect the bottom portion (as shown) downward, driving the microneedles down; the force applied by the bias 211 may be greater than the restoring force provide by the bottom cover portion. The bottom cover may be an elastic band or gasket around the perimeter of the mount, and may include an opening or openings for the microneedles or may pass around them, or may be pierced by the microneedles. As will be described in more detail below, the bias may be triggered (e.g., by pushing on the front surface of the holder or a button on the front surface) to drive the microneedles down, e.g., into the gingiva.

FIG. 2C shows another example of a mount 205″ for a soft-tissue sensor, or a portion of a soft tissue sensor including a microneedle, similar to that shown in FIGS. 2A-2B. In FIG. 2C, the cover may be removable or integral with the mount. The soft-tissue sensor in this example includes a single microneedle that extends down, e.g., towards or into the gingiva.

In any of these variations, the soft tissue sensor may include a probe, rather than, or in addition to, a microneedle. For example, the soft tissue sensor may include one or more electrical probes that extends down to or into the gingiva, in order to transmit and/or receive electrical signals from the tissue. Alternatively, the microneedle may be configured as a probe.

Alternatively or additionally, the bias may be configured to permit a microneedle/probe to push up on the sensor, without penetrating the soft tissue, while maintaining the probe in good (e.g., electrical) contact with the tissue. For example the bias on the upper side of the mount may provide only a very low level of force, so that the pressure on the microneedle or probe may be less that that required to penetrate the tissue. FIG. 2D shows an example of a mount 205″' that is configured to “float” the non-penetrating microneedle (configured as a blunted probe 209′ having an enlarged, rounded tissue-engaging bottom) against the gingiva. The mount in this case include the soft biasing element 211′ and a deformable (e.g., in this example, elastically deformable) bottom 213′.

In some variations, the sensor itself (without necessitating a separate ‘probe’ or needle portion) may be drive against the soft tissue when held by the intraoral appliance. For example, the senor may include a sensing surface (e.g., electrode, etc.). The bias on the intraoral soft tissue sensing system may drive the sensor against the soft tissue.

The soft tissue sensors shown corresponding to the mounts shown in FIGS. 2A-2D are exemplary only. The shape and size of the soft tissue sensor may be different. The mount may be configured to hold the entire sensor, or a portion of the sensor, and particularly the portion including the microneedles, and may include a bias, as shown. Alternatively, the soft tissue sensor may be held in multiple mounts, any of which may include a bias. In some variations the mount may apply the biasing force to the soft-tissue sensor and/or to the microneedles specifically.

In general, any of the sensors described herein may include a non-penetrating microneedle, which may be referred to as a probe, that is configured to be in contact with the soft tissue without penetrating the tissue. FIG. 2D, described above, shows on example of this. Any number of such non-penetrating microneedles (probes) may be used for a particular sensor, and the probe may be configured to contact the surface of the tissue without penetrating. The sensor may be configured to include multiple sensing features, including electrical (e.g., conductance, resistance, etc.), mechanical (vibration, deflection, etc.), chemical, thermal (temperature sensing, etc.), or the like.

In any of these variations, the outer surfaces of the mount may be configured to form a smooth and/or continuous surface with the intraoral appliance. Any of these mounts may be formed integrally with the intraoral appliance, or they may be separately attachable to the intraoral appliance.

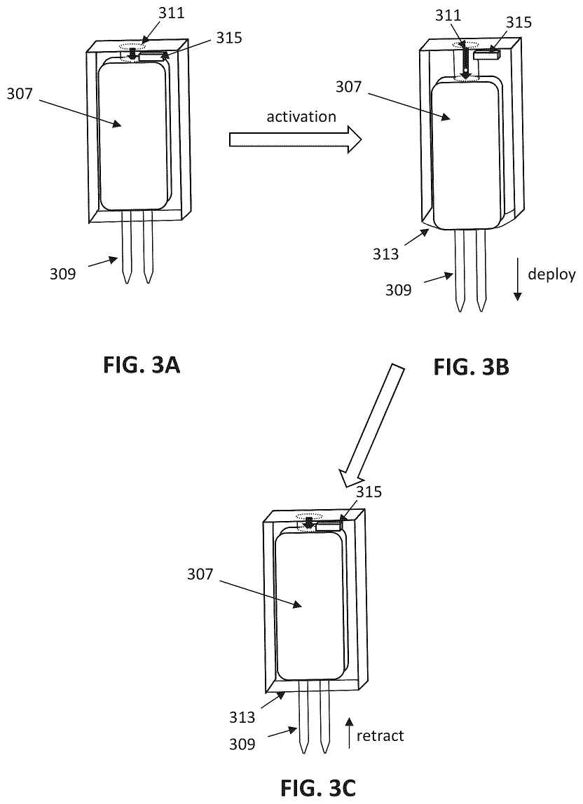

As mentioned above the biasing in any of these variations may be configured to be activatable, so that a basing force (e.g., driving the microneedle(s) and/or sensors down into or onto the tissue) may be applied only after activating the device, e.g., when the device has been seated onto the patient's teeth. FIGS. 3A-3C illustrate an example of an activatable bias. In this example, similar to the variation shown in FIG. 2B, above a sensor 307 is shown in the mount 305. Initially, the bias 311 is not engaged, and is not applying a force against the senor/microneedles 309 (see FIG. 3A). The mount may include a cover (not shown). In this example the bottom surface of the mount may also be formed of an elastically deformable material, as described in FIG. 2B. A deployment control (e.g., button, trigger, switch, etc.) may be included as part of the mount. In FIG. 3A, the deployment control is shown as a button 315 on the face or front of the mount (e.g., on the cover or outer surface). Alternatively, the deployment control may be electronic/electric and may receive an activation signal remotely (e.g., from an application software in communication with the sensor and/or mount), or it may be located on an inner surface of the mount or within the cavity (including near the occlusal surface of the cavity, so that the bias will be automatically deployed when the apparatus is seated on the teeth. In FIG. 3A, the bias may be deployed (e.g., released) by pushing on the button 315, allowing the bias to activate, as shown in FIG. 3B. In FIG. 3B, the bias 311 is pushing against the elastically deformable bottom 313 of the mount, driving the microneedles 309 down into a deployed configuration. Note that the extent of deployment (e.g., the length that the microneedles are extended by the bias) may be any appropriate length, which may be set by choosing the geometry of the bias, as well as the elastic properties of the bottom of the mount.

The microneedles may be retracted by disengaging the bias 311, as shown in FIG. 3C. The bias may be disengaged by another control, or by the same control 315, which may retract the needles, or allow them to retract based on the elastic properties of the bottom of the mount. For example, the bias may be retracted by moving it up, away from the sensor, by collapsing it back down, or by otherwise releasing the biasing force.

In general, any appropriate bias may be used, including mechanical (e.g., spring, coil spring, leaf spring, etc.), electrical/magnetic (e.g., solenoid, electromagnet, etc.), pneumatic, liquid, etc. The type of bias chosen may determine the activation/inactivation means used.

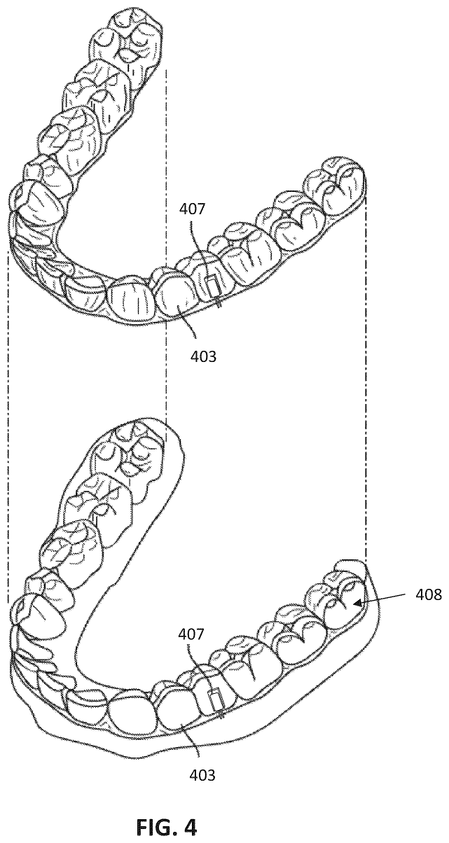

FIG. 4 illustrates the application of an intraoral, soft-tissue sensor system onto a subject's teeth 408. A sensor 407 is mounted into the intraoral appliance forming the intraoral, soft-tissue sensor system 403, and the teeth fit snugly into the cavity formed by the intraoral appliance, which is shown as transparent in this example. The apparatus may be worn so that the microneedles of the sensor are inserted into or onto the gingiva, as shown, to provide monitoring, including continuous monitoring for one or more analyte, depending on the sensor configuration. The methods and apparatuses described herein may be generic to a variety of different sensors, and may provide a platform for stably holding a sensor having a probe or microneedle relative to the gingiva, by connecting to the hard tissue of the patient's teeth. The intraoral, soft-tissue sensor systems described herein may therefore provide a comfortable platform for measuring from the soft tissue.

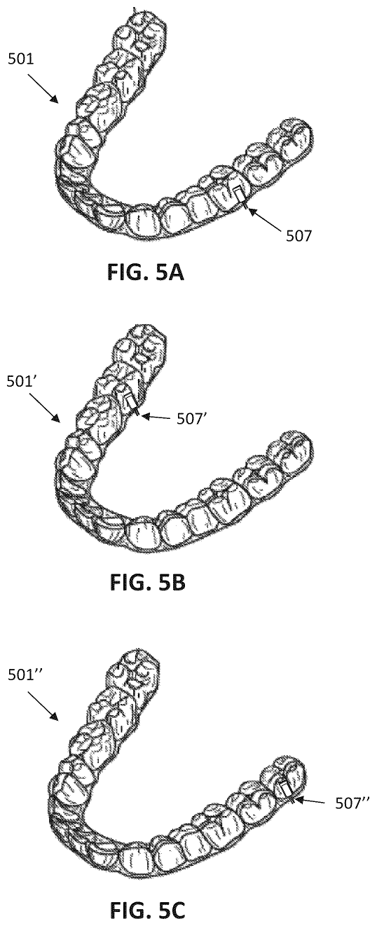

Long term monitoring may be possible with any of the intraoral, soft-tissue sensor systems described herein. In particular, any of the methods and apparatuses described herein may include a series of intraoral, soft-tissue sensor system that may be worn sequentially, as shown in FIGS. 5A-5C. In this example, the sensor 507 may be mounted at different locations. Thus, at different times, the microneedle/probe(s) of the sensor may be interacting with different regions in the oral cavity soft tissue. This may reduce or eliminate irritation and prevent harm to the tissue. For example, the first apparatus 501 may be worn for a first time period (1 hour, 2 hours, 3 hours, 4 hours, 6 hours, 12 hours, 24 hours, 36 hours, 2 days, 3 days, 4 days, 5 days, 6 days, 1 week, 2 weeks, 3 weeks, etc., preferably 1-3 days), the switched and the second apparatus 501′ may be worn. The sensor in the second apparatus is on a different portion of the intraoral appliance. Again, after a second time period, a third apparatus 501″ may be worn. This process may be repeated with as many apparatuses as desired. The apparatuses may be reused (e.g., as part of a repeating cycle), with cleaning between use. The patient may insert and remove the devices themselves, without the need for a physician or technician.

Alternatively, the same apparatus, which may be configured to move the mount for the sensor and/or may have a plurality of mounts on the intraoral appliance body, may be used. After the desired wearing time period, the apparatus may be removed from the patient's mouth and the sensor(s) moved to a different location on the apparatus.

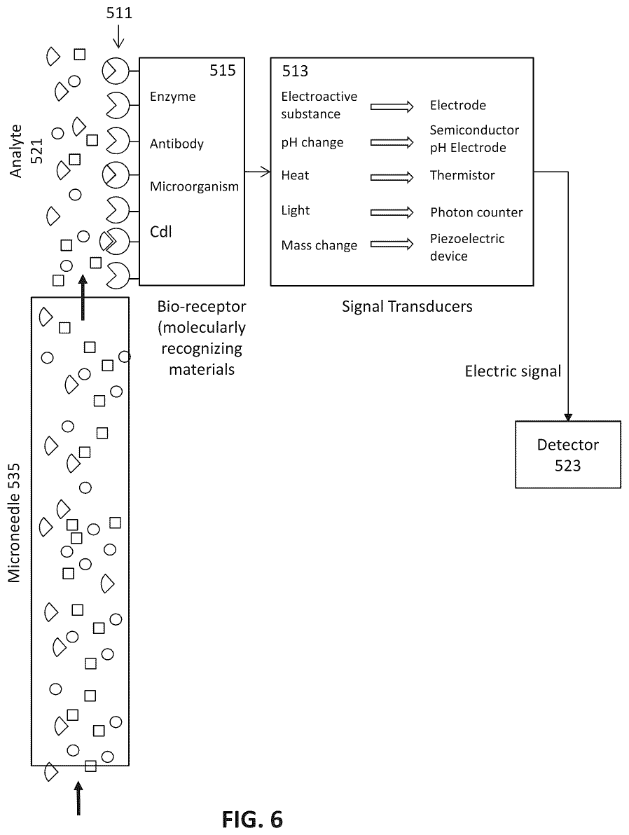

As mentioned above, any appropriate sensor may be used. For example the sensor may be configured with a hollow microneedle (or multiple hollow microneedles), a bio-receptor portion to bind and/or otherwise interact with a target analyte and a signal transducer to convert the binding/interaction into a discernable signal that may be converted into an electrical output and sent to a detector, for further processing. FIG. 6 illustrate a schematic example of such a sensor. In this example, the microneedle 535 is hollow and allows passage of a fluid (e.g., blood, lymph, interstitial fluid, etc.) containing one or more analyte 521 into a sensing region 515 that include a bio-receptor 511 that interacts with (e.g., binds) the analyte and triggers a signal transduction event. For example, the bio-receptor may be an enzyme, antibody, microorganism, Cdl, etc. that may alter its properties (including optical, chemical, electrical, or other measurable properties) or trigger a detectable change 513 that transduces the interaction event.

Any of the apparatuses described herein may be configured as intraoral (or “oral”) sensors that may be held in continuous contact with the soft tissue, such as the gums (gingiva), and/or palate. These apparatuses may include or be configured for use with one or more microneedles that maybe immersed in the tissue or may contact the tissue. Microneedles may be used to measure passively, deliver signals actively, and/or deliver and extract materials from and to the body. Typically, the sensor may be held in place via an appliance attached to the teeth. A bias, such as a spring (or spring-like) mechanism may be used to apply a continuous force on the senor to keep the sensor in place, as described above. The sensor may be detachable (from the appliance) and may optionally be configure so as to not interfere with the patient's bite. Additionally the apparatus, including the sensor or a portion of the sensor may be formed of a transparent and/or translucent material. Any of these apparatuses may be configured so that they may be supported in the mouth using a mini implant (e.g., TAD), and/or a screw to a prosthetic tooth, and/or an attachment on one or more of the patient's teeth.

For example, any of the apparatuses described herein may be configured to actively apply energy (e.g., current) to the soft tissue via one or more contacts, including the microneedle/probes described herein. Thus, a sensor included as part of the intraoral, soft-tissue sensor systems described herein may be configured to deliver/transmit a stimulation, including (but not limited to) electrical stimulation. For example, a sensor may be configured to transmit a low level current to the soft tissue. A current may be applied, for example, to activate the patient's senses and/or to directly activate the patient's muscles. This may be therapeutically useful to treat snoring or apnea in variations configured to detect snoring or apnea, as it may encourage or cause the airways of a sleeping patient to open, e.g., when snoring. Other materials, including drugs, or active chemical agents, may be released by the apparatus in response to a sensed condition. One or more drugs could be delivered from the same needle (e.g., microneedle) used for sensing, or a separate one or more microneedles could be used. For example, a sensor configured to detect a patient condition such as insulin level in a diabetic patient wearing the device could be administered a small amount of insulin through the same of a different microneedle in contact with the soft tissue within the oral cavity. This may allow both continuous glucose monitoring (e.g., via a sensor and microneedle(s)) and potentially may allow immediate delivery of insulin via the same apparatus. Alternatively, the intraoral system may communicate with (or include) a separate drug pump for delivery of drug (e.g., insulin, etc.).

Any of the apparatuses described herein may be configured to deliver a drug, including in particular, delivering a drug based on a sensed need for the drug.

In addition, the apparatuses described herein may be configured to sense from other portions of the patient's oral cavity, in addition or instead of the soft tissue. For example, the sensors used may include sensors configured to measure or detect air flow due to inhaling and exhalation, and/or may analyze the inhaled gas and warn for toxic or harmful substances, and/or may analyze air exhaled by the patient and warn for issues that may be detected by sensed analytes, and/or analyze breath rates, pacing, etc., and/or detect and warn of halitosis, and/or detect alcohol level.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

For example, any of the sensors described herein may be configured to communicate in one-way or two-way communication with a remote (e.g., external) device, such as an external processor (e.g., smartphone, computer, wearable electronics, pad, etc.). Alternatively or additionally, any of these sensors may be configured to communicate with a processor on or in the intraoral appliance. The communication may be via a wired connection or a wireless connection; the processor in/on the intraoral appliance may store, modify, and/or send/receive data from the sensor. Thus any of these sensors and/or components in the intraoral soft tissue sensor system (including a processor or processors) may include wireless communication circuitry (e.g., WiFi, Bluetooth, Zigbee, etc.). Any of these apparatuses may also include power (e.g., battery, power control circuitry, inductive power/charging, etc.).

### EXAMPLES

Any of the soft tissue sensors including one or more microneedles described herein may be used with or integrated into an orthodontic apparatus as described herein and may include a sensor (e.g., biosensor) configured to be positioned in a subject's oral cavity, including but not limited to a soft tissue, and an electronics system in communication with the sensor, where the electronic system includes one or more of: a signal amplifier, a signal conditioner (e.g., filter, averaging, etc.), processor, memory (e.g., data logging unit), power source (e.g., battery, capacitor, etc.) and data communications (e.g., wired or wireless communications, such as Bluetooth, Wifi, Zigbee, RFID, ultrasound, or the like). The sensor may convert a biomarker detection into an electrical signal, and may include a bioreceptor and a biotransducer. The bioreceptor may be configured to interact with a specific analyte or subset of analytes and produce a measurable signal (optical, chemical, mechanical, thermal, electrical, or some combination of these) that is transduced by the biotransducer into an electrical signal that can be processed (amplified, filtered, averaged, combined, etc.) by the electronic subsystem. The electronic subsystem may be an integrated system (e.g., a CMOS-based microprocessor). All or some of these components may be on or part of an oral appliance, such as an aligner, brace, palatal expander, etc.

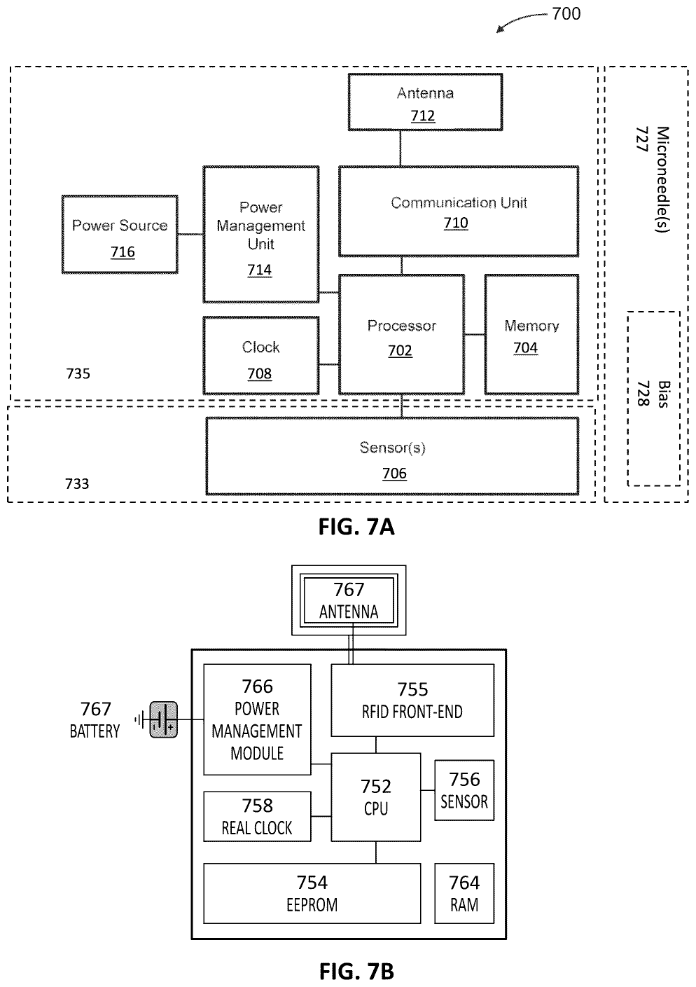

For example, FIG. 7A shows a portion of an apparatus that includes a biosensor (sensor(s) 706) comprising a bioreceptor and biotransducer, in communication with an electronics system 735. The biosensor may be integrated and/or incorporated with the electronics system 735 and connected directly to a patient, etc., on a tooth, teeth, gingiva, etc., or it may be connected to an oral appliance. The biosensor(s) 733 may be separate from the electronics system 735 (indicated by the dashed lines. For example, the biosensor(s) may be part of a second oral appliance and/or directly mounted in the oral cavity (e.g., on a tooth/teeth), and may couple to an oral appliance including all or some of the electronics system (e.g., battery/power source 716, processor 702, antenna 712, memory 704, etc.). The biosensor may include any of the microneedles projecting from the intraoral appliance that are configured to penetrate soft tissue adjacent to the patient's teeth when worn and in some variation a bias coupled to the soft tissue sensor configured to apply a biasing force to the soft tissue sensor. The microneedles may be part of the biosensor 733 or part of the electronics system 735, or both.

FIG. 7B is another example of a schematic illustrating a biosensor apparatus. In FIG. 7B, the biosensor(s) 756 are integrated with the electronics system. The electronics system in this example include a processor (CPU 752) that may amplify, filter, analyze and/or store the signals from the biosensor(s). The biosensor(s) may include a bioreceptor and a biotransducer that converts interaction (e.g., binding, enzymatic interaction, etc.) with a biomarker into an electrical signal. The electronics may further include an oscillator/clock 758, and a memory (e.g., EEPROM 754). Additional memory (e.g., RAM 764) may also be included. The memory may store data generated by the biosensor(s) and/or control logic/command logic for operating the apparatus. This logic may be modified (e.g., programmable). Additional memory the electronics system (which may also be referred to herein as a sensor electronics system or sub-system) may include communications circuitry (shown as an RFID front end 755) in FIG. 7B, which may also include or communicate with an antenna 757. The electronics system may also include power management circuitry 766, for regulating the power used by the electronics and/or biosensor(s). The power management circuitry may regulate a power source, such as a battery 767.

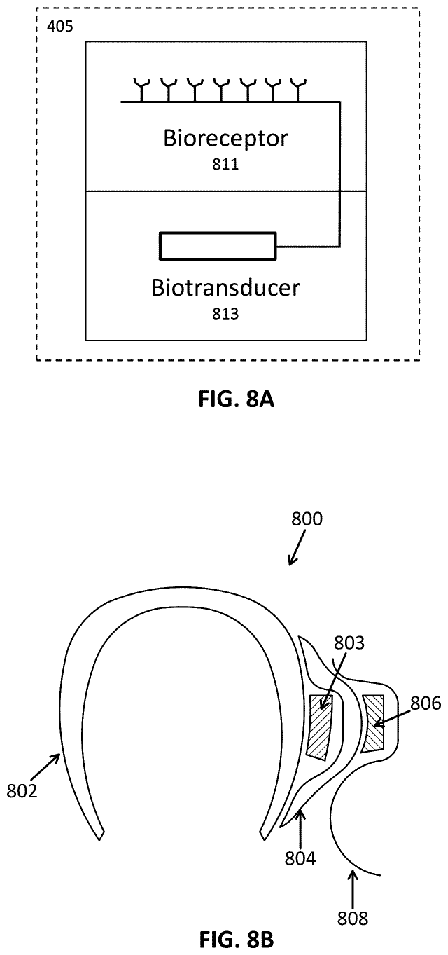

As schematically illustrated in FIG. 8A, a biosensor 405 may include a bioreceptor 811 that interacts with a biomarker and is functionally coupled with a biotransducer 813. In this example, the bioreceptor is shown schematically including specific interaction sites that may engage with a biomarker, e.g., by binding, enzymatically reacting with, etc. The biotransducer 813 is functionally linked to bioreceptor and may transduce interaction between the biomarker and the bioreceptor into an electrical signal (output) that is passed on to the electronic system (not shown, sec, e.g., FIGS. 7A and 7B). For example, the biotransducer may bind to the bioreceptor and binding may be detected by the biotransducer via an optical signal. In some variations, the bioreceptor may be bound to an optically transparent substrate through which light may be passed by the biotransducer; a change in the optical properties of the bioreceptor may correlate with binding of the biomarker to the biotransducer. Alternatively, the bioreceptor may include a matrix (e.g., hydrogel) that interacts with the biomarker to modify a property of the matrix (e.g., electrical resistance, optical absorption, electrochemical potential, etc., and this modified property may be polled and/or detected by the biotransducer. Other specific examples are provided below. A microneedle may project from the intraoral appliance and/or from biosensor.

Any appropriate biomarker or biomarkers may be used. Biomarkers may be biomolecules or byproducts of biomolecules that are present in the oral cavity (including saliva, GCF, and/or breath) and/or contaminants that may be present in the oral cavity, such as bacteria, yeast, etc. Biomolecules of particular interest include those that change in response to movement of the teeth due to an orthodontic procedure. For example, Table 1, below lists examples of biomarkers that may be tested using the biosensor apparatuses described herein. For example in Table 1, protein biomarkers include Protein S100-A9 (e.g., S100 calcium-binding protein A9, Calgranulin-B), Serum albumin precursor, Immunoglobulin J chain, Ig alpha-1 chain C region, Cysteine-rich secretory protein 3 precursor (CRISP-3), Hemoglobin subunit beta (Hemoglobin beta chain, Beta-globin), and 14-3-3 protein σ (Stratifin, Epithelial cell marker protein 1).

TABLE 1
Summary of saliva proteins with expression change during orthodontic treatment. (These are proteins that
show statistically significant change (P < 0.5) between day 0 and day 14 of orthodontic treatment.)

| | | | Accession | | | | | |
|---|---|---|---|---|---|---|---|---|
| | | | number | MW (kDa)/pI | | Peptides matched/% | | Expression |
| Spot no. | Protein name | Known Function | (UniProt) | Theoretical | Experimental | coverage (Protein score) | | change |
| 1 | Protein S100-A9 | (i) Calcium-binding protein. | P06702 | 13.2/5.71 | 14.5/5.47 | 4/24% | (121) | Down regulated |
| | (S100 calcium- | (ii) Promotes phagocyte | | | | | | at day 14 |
| | binding protein | migration and infiltration of | | | | | | |
| | A9) (Calgranulin-B) | granulocytes at sites of | | | | | | |
| | | wounding. | | | | | | |
| | | (iii) Plays a role as a | | | | | | |
| | | proinflammatory mediator | | | | | | |
| | | in acute and chronic | | | | | | |
| | | inflammation. | | | | | | |
| 2 | Serum albumin | (i) Good binding capacity | P02768 | 69.3/5.92 | 14.8/7.00 | 8/11% | (105) | Down regulated |
| | precursor | for water, Ca2+, Na+, K+, | | | | | | at day 14 |
| | | fatty acids, hormones, | | | | | | |
| | | bilirubin, and drugs. | | | | | | |
| | | (ii) Main function is the | | | | | | |
| | | regulation of the colloidal | | | | | | |
| | | osmotic pressure of blood. | | | | | | |
| | | (iii) Major zinc transporter | | | | | | |
| | | in plasma. | | | | | | |
| 3 | Immunoglobulin J | (i) Serves to link two | P01591 | 15.5/4.62 | 34.0/3.86 | 10/30% | (220) | Down regulated |
| | chain | monomer units of either | | | | | | at day 14 |
| | | IgM or IgA. | | | | | | |
| | | (ii) Help to bind IgM or IgA | | | | | | |
| | | to secretory component. | | | | | | |
| 4 | Immunoglobulin J | | P01591 | 15.5/4.62 | 36.4/3.40 | 6/27% | (198) | Down regulated |
| | chain | | | | | | | at day 14 |
| 5 | Ig alpha-1 chain C | (i) Major immunoglobulin | P01876 | 37.6/6.08 | 44.76/6.91 | 9/20% | (260) | Down regulated |
| | region | class in body secretions. | | | | | | at day 14 |
| | | (ii) Serve both to defend | | | | | | |
| | | against local infection and | | | | | | |
| | | to prevent access of foreign | | | | | | |
| | | antigens. | | | | | | |
| 6 | Cysteine-rich | (i) Innate immune response | P54108 | 27.6/8.09 | 43.25/8.90 | 2/6% | (94) | Present only |
| | secretory protein 3 | (ii) Potential biological | | | | | | at day 14 |
| | precursor (CRISP-3) | marker for prostate cancer | | | | | | |
| 7 | Hemoglobin | Involved in oxygen transport | P68871 | 15.9/6.75 | 14.83/9.50 | 6/27% | (158) | Present only |
| | subunit beta | from the lung to the various | | | | | | at day 0 |
| | (Hemoglobin beta | peripheral tissues. | | | | | | |
| | chain) (Beta-globin) | | | | | | | |
| 8 | 14-3-3 protein σ | (i) Adapter protein. | P31947 | 27.7/4.68 | 44.76/4.20 | 5/22% | (147) | Present only |
| | (Stratifin) (Epithelial | (ii) Binds to a large number | | | | | | at day 0 |
| | cell marker protein 1) | of partners, generally results | | | | | | |
| | | in the modulation of the | | | | | | |
| | | activity of the binding | | | | | | |
| | | partner. | | | | | | |

Table 1: Gingival Crevicular Fluid (GCF) biomarkers (Table 1 is adapted from Ellias M F et al. “Proteomic Analysis of Saliva Identifies Potential Biomarkers for Orthodontic Tooth Movement”. The Scientific World Journal. 2012; 2012: 647240. doi: 10.1100/2012/647240.)

Any of the apparatuses and methods described herein may target biomarkers present in gingival cervicular fluid (GCF), which may be collected by any of the microneedles described herein. In general, various molecules are capable of passing through gingival sulcular epithelium and may filter into GCF. Many of these molecules are associated with remodeling of paradental tissues during situations such as normal maintenances, periodontal diseases and Orthodontic treatment. The collection and analysis of GCF is a non-invasive procedure that may be useful information on the nature and extent of the periodontal response to mechanotheraphy and orthodontic treatments. The apparatuses and methods described here may be configured and/or adapted to collect GCF. For example any of the apparatuses described herein may include one or more microneedles projecting and extending between the teeth and gingiva (e.g., penetrating to a depth of 0.5 mm, 1 mm, 2 mm, 3 mm, 4 mm, 5 mm, 6 mm, etc.). The microneedle may be fixed or extendable as described above.

Modified or enhanced cellular activities during orthodontic tooth movement can be found in GCF of treated teeth. Additional biomarkers that may be examined, e.g., in GCF and/or saliva include prostaglandin E (PGE) (elevated prostaglandin E (PGE) levels in GCF 1 day after application of mechanical stimuli has been detected), cytokines, including IL-6 and IL-8, TNF-α, Hyaluronic acid, chondroitin sulphate, IGF, Acid phosphatase, Aspartate aminotransferase, Alkaline phosphatase (ALP), Lactate dehydrogenase, Collagenase, Matrix metalloproteinases (e.g., MMP-1, MMP-2 and MMP-8), Cathepsin B, TRAP, Osteocalcin, Osteonectin, Osteopontin and dentin sialoprotein. For example, a correlation has been found between the velocity of tooth movement and increase in concentrations of cytokine and its receptor antagonist. IL-6, IL-8 levels in GCF after force application has been shown. Increased level of TNF-α in GCF after force application which peaked at day 1 has been shown. Studies demonstrated elevated Hyaluronic acid in all GCF samples and chondroitin sulphate levels in GCF increased greatest in teeth that moved most. IGF (bone remodeling marker) may be elevated, and its binding protein levels in GCF, 4 h after mechanical stimulation. Acid phosphatase and Aspartate aminotransferase levels after force application are higher on compressed side compared to tension side in GCF. Alkaline phosphatase (ALP) levels are higher on tension side compared to compression side. Lactate dehydrogenase levels are higher on compression side whereas Collagenase levels are elevated on both mesial and distal sides after mechanical stimulus. Matrix metalloproteinases (MMPs) (MMP-1, MMP-2 and MMP-8) show elevated levels on compressed side than on tension side. Elevated levels of Cathepsin B, an indicator of ECM degradation, were demonstrated in GCF 1 day after force application. Elevated levels of TRAP in GCF on the compression side after force application has been shown. Osteocalcin, a bone turnover marker, may be elevated in GCF of patients with periodontal breakdown. Elevated levels of Osteonectin and Osteopontin have been detected in GCF with progressive increase in periodontal breakdown. Elevated levels of dentin sialoprotein in GCF samples of teeth at 12 weeks following commencement of fixed appliance therapy have also been demonstrated.

For example, the apparatuses and methods described herein may be configured to detect one or more of the following biomarkers. In some variations, these biomarkers may be detected from the saliva and/or the gingival crevicular fluid (GCF). These biomarkers may be particularly helpful in detecting tooth remodeling or movement. The levels of one or more of these biomarkers may be detected and tracked over the course of a treatment to adjust or modify an orthodontic treatment. For example, one of more markers for inflammation, remodeling and/or enzymes (e.g., enzymes associated with bone resorption, formation, cell necrosis, collagen remodeling, etc.) may be detected. Examples of makers for remodeling of the teeth may include: Glycosaminoglycans (e.g., in GCF), including hyaluronic acid and a minor band of chondroitin sulfate, Pyridinium derivatives (e.g., pyridinoline and deoxypyridinoline), Pentraxin-3, also known as tumor necrosis factor (TNF)-stimulated gene 14 (TSG-14), N-telopeptide type 1 and osteocalcin, Osteocalcin, and Matrix metalloproteins (MMPs) 1 and 8. Markers for inflammation may include: Prostaglandin E (PGE2), Neuropeptides (calcitonin related gene peptide and substance p), Transforming growth factor-α1, Epidermal growth factor (EGF), α2 Microglobulin (α2MG), insulin-like growth factor-1, Interleukin-1 (receptor antagonist) (IL-1) 1β, 2, 6, 8 cytokines, Tumor necrosis factor-α, Macrophages colony stimulating factors, TNF-related ligand receptor activator of nuclear factor-kappa ligand (RANKL) and its two receptors, receptor activator of nuclear factor-kappa (RANK), and osteoprotegerin (OPG), and Myeloperoxidase (MPO). Markers of root resorption may include: dentine matrix protein 1, dentin phosphoprotein (DPP), and dentin sialoprotein (DSP). Enzymes and enzyme inhibitors may include: Cathepsin B, Acid phosphatase (ACP) and alkaline phosphatase (ALP), β-Glucuronidase (βG), Aspartate aminotransferase (AST), and lactate dehydrogenase.

As mentioned above, any of the biosensors described herein may include a bio-recognition component, a biotransducer component, and electronic system which may include a signal amplifier, processor, data logging units and data communication unit. In some instances, transducers and electronics can be combined such as in CMOS-based microsensor systems. The recognition component may be called a bioreceptor, may use a biomolecule from organisms or receptors modeled after biological systems to interact with the analyte of interest. This interaction may be measured by the biotransducer which outputs a measurable signal proportional to the presence of the target analyte in the sample. The processor can log the raw or processed data in the memory unit or transmit it to a receiver. The system can work actively if energized with battery, super-capacitor, or an energy harvesting unit, or it may perform passively upon being energized via induction using an external device, such as cell phone.

In any of the biosensors described herein, the bioreceptor may be configured to interact with the specific analyte of interest to produce an effect measurable by the transducer. The bioreceptor may have a high selectivity for the analyte among a matrix of other chemical or biological components. While the type of biomolecule used may vary widely, biosensors may be classified according to common types of bioreceptor interactions involving, e.g., interactions such as: antibody/antigen, enzymes/ligands, nucleic acids/DNA, cellular structures/cells, or biomimetic materials. The bioreceptor may be configured to engage in one or more of these interactions (e.g., may include a bound or engineered antibody, enzyme, nucleic acid sequence, protein or engineered protein, etc.) in a localized manner that may be interrogated by or communicated to the biotransducer.

For example, a biosensor as described herein for use with an oral appliance may be configured to take advantage of an antibody/antigen interaction with one or more of the biomarkers described herein. Thus, the biosensor may be configured as an immunosensor. An immunosensor may utilize the very specific binding affinity of antibodies for a specific compound or antigen. The specific nature of the antibody antigen interaction is analogous to a lock and key fit in that the antigen will only bind to the antibody if it has the correct conformation (proper selection of primary and secondary antibodies). Binding events result in a physicochemical change that, in combination with a tracer, such as fluorescent molecules or enzymes, can generate a signal such as an elevation in voltage that can be detected by electronic components. Binding may be detected optically (e.g., by color, or transmission) and/or electrically.

Also described herein are biosensors that include enzymatic interactions. For example, analyte recognition may be enabled through: (1) an enzyme converting the analyte into a product that is sensor-detectable, (2) detecting enzyme inhibition or activation by the analyte, or (3) monitoring modification of enzyme properties resulting from interaction with the analyte. Since enzymes are not consumed in reactions, the biosensor may be used continuously. The catalytic activity of enzymes may also lower limits of detection compared to common binding techniques.

Other biosensing techniques that can be used may include detecting nucleic acid interactions, detecting epigenetic modifications, cell based, and tissue based detection.

As mentioned above, a biotransducer may be electrochemical, optical, electronic, piezoelectric, gravimetric, and/or pyroelectric-based.

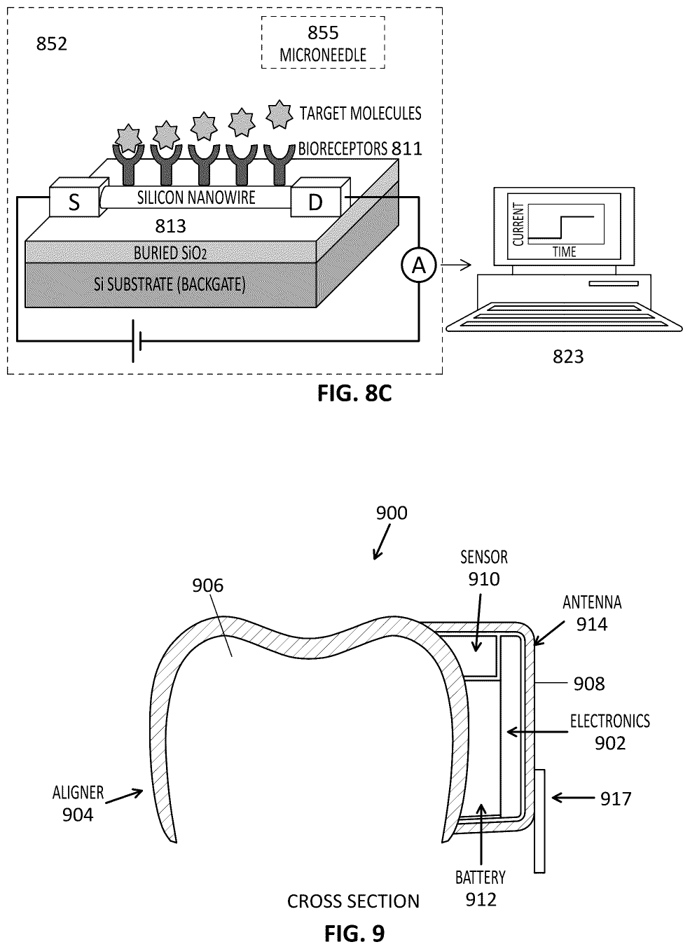

FIG. 9 illustrate an example of an apparatus including a biosensor as described herein, similar to that shown in FIGS. 1A and 1B. For example, In FIG. 9, the apparatus 900 includes an orthodontic device, shown as an aligner 904 to which a biosensor or biosensors are coupled, either integrated into the housing of the electronics sub-assembly 902, or separate 951 but electrically connected to the electronics. The electronics (processor, battery, communications circuitry, antenna, etc.) is shown in a housing that mounted to an outside portion of the aligner, shown on the buccal side, although it may be on the lingual side instead or additionally. FIG. 9 shows an example of a section through an apparatus in which the biosensor (or a separate sensor 910, such as a temperature sensor or the like) is included with the electronics 902. An outer cover 908 may hold the assembly to the aligner 904 and the apparatus may be placed over the teeth 506. One or more microneedle 917 may extend or be extendable from the device. In some variations the electronics and/or sensor are on the inner surface of the aligner (e.g., the cavity into which the teeth sit).

Although the examples shown above indicate that the microneedle(s) are extending from an outer surface of the aligner, in some variations some or all of the microneedles may extend from an inner surface of the aligner and into the gingiva or may sample from the saliva above the gingiva. In particular, the one or more microneedles may be configured to sample from the space between the patient's teeth.

In addition to the biosensors described above, also described herein are sensors configured to determine stress-induced bioelectric potentials on the teeth. Stress-induced bioelectric potentials may regulate alveolar bone remodeling during orthodontic tooth movement. For example, the force, F, applied to the labial surface of the lower incisor that displaces the tooth in its socket, deforming the alveolar bone convexly towards the root at the leading edge, may produce concavity towards the root at the trailing edge. Concave bone surfaces characterized by osteoblastic activity are electronegative; convex bone surfaces characterized by osteoclastic activity are electropositive or electrically neutral. Measuring the electrical charge on the teeth surface, may be used to determine the tooth movement rate and direction. Such data can be used to conduct a closed-loop orthodontic treatment. For example, any of the methods an apparatuses described herein may measure or detect the electrical charges from the surface of different regions of a subject's teeth. An apparatus may include a plurality of electrodes within the concavity of an aligner, or a plurality of electrical contacts for contacting electrodes attached to the teeth, e.g., attached to one or both of the buccal and lingual sides of any of the teeth that are being moved by the orthodontic appliance. The apparatus may include the electrical system that is configured to receive these surface charge readings and may store, transmit (e.g., wireless) or analyze these signals, e.g., in the electrical system or remotely, to determine and/or evaluate forces on the teeth and tooth movement.

Also described herein are systems and apparatuses for determining one or more indicator of the patient's heath. For example, any of these methods and apparatuses may include a sensor configured as a physiological sensor to detect one or more subject physiological state. For example, any of the apparatuses or methods described herein may measure (e.g., using a physiological sensor) one or more of: electrocardiogram (ECG), bio-impedance, blood oxygenation, galvanic skin response, heart rate, body temperature, respiration (including respiration rate), or the like. For example, any of these apparatuses may include an ECG sensor (e.g., one-point ECG electrode), a thermistor, a bio-impedance sensor, a photoplethysmogram sensor, a galvanic skin response sensor, etc., including electronics to support such sensor(s). These sensors can be utilized by themselves or with any other sensor, including one or more of the biosensors described herein. As with any of the biosensors and sensors described herein, these sensors may be used to detect or determine compliance (e.g., use of the aligners) while they generate health information of the patient. For instance, a photoplethysmogram (PPG) sensor may measure blood-volume changes in the blood tissue. A plethysmogram is volumetric measurement of an organ. This technique is non-invasive and may be obtained by illuminating light into the body and measuring the change in light absorption. In the current invention, this technique may be applied within the intraoral cavity. A plethysmography sensor can be incorporated in an orthodontic appliance (e.g., aligner) to determine the blood volume change in a check or within an extended gingiva segment near or under the appliance to detect blood volume change in gingiva.

Alternatively or additionally, a galvanic skin response (GSR) sensor may be used to measure conductivity of intraoral tissues. Conductivity may change with both changes in the underlying amount of minerals released onto the outer surface of tissues from glands.

Also described herein are methods and apparatuses for detecting and/or analyzing breath. For example any of the apparatuses described herein may be used to detect and/or diagnose disease. For instance, lung and breath cancers may be detected via analysis of the breath, e.g., by identifying particular breath volatile organic compounds (BVOCs) that differ between patients with non-small cell lung cancer (NSCLC) and subjects without the disease. Other sensors that detect cancer at early stages via, for instance, measuring chemical components of breath during exhale. Exhaled breath contains both volatile and non-volatile organic compounds, which vary between healthy individuals and those with lung cancer.

The apparatuses and methods described herein may also be configured to detect halitosis (bad breath). Halitosis may arise from inside the mouth and/or due to a disorders in the nose, sinuses, throat, lungs, esophagus, or stomach. Bad breath may also be due to an underlying medical condition such as liver failure or ketoacidosis. Halitosis may also arise from an underlying disease such as gum disease, tooth decay, or gastroesophageal reflux disease.

By far the most common causes of halitosis are odor producing biofilm on the back of the tongue, below the gum line, and in the pockets created by gum disease between teeth and the gums. This biofilm results in the production of high levels of foul odors produced mainly due to the breakdown of proteins into individual amino acids, followed by the further breakdown of certain amino acids to produce detectable gases. Volatile sulfur compounds are associated with oral malodor levels, and usually decrease following successful treatment. The intensity of bad breath may differ during the day, due to eating certain foods (such as garlic, onions, meat, fish, and cheese), smoking, and alcohol consumption. The odor may be worse upon awakening and may be transient or persistent (e.g., chronic bad breath).

The apparatuses and methods described herein may be configured to detect one or more compounds or markers for bad breath (e.g., above a target threshold) that may indicate bad breath, and may alert the wearer, track, store, and/or transmit detected levels. Markers that may be detected by the apparatuses described herein may include indole, skatole, polyamines, volatile sulfur compounds (VSCs) such as hydrogen sulfide, methyl mercaptan, allyl methyl sulfide, and dimethyl sulfide. In some variations the apparatus may detect one or more bacterial markers arising due to halitosis-producing bacteria. Bacteria that cause gingivitis and periodontal disease (periodontopathogens) may be gram negative may produce VSC. Methyl mercaptan is known to be a contributing VSC in halitosis that and may be caused by periodontal disease and gingivitis.

These apparatuses may also be used to determine, detect and/or diagnose other dental issued, including gum disease. For example, the level of VSC on breath has been shown to positively correlate with the depth of periodontal pocketing, the number of pockets, and whether the pockets bleed when examined with a dental probe. VSC may themselves contribute to the inflammation and tissue damage that is characteristic of periodontal disease. Markers for halitosis may also suggest or indicate infection (e.g., oral infection), oral ulceration, stress/anxiety, menstrual cycle (e.g., at mid cycle and during menstruation, increased breath VSC has been reported), or the like. Any of the methods and apparatuses described herein may also or alternatively be used to detect or determine (and/or aid in treatment) of any of these indications.

For example, a dental apparatus including a sensor may be configured to detect sulfide (e.g., sulfur emissions) from the patient's breath, saliva and/or GCF. For example, the sensor may be configured to detect hydrogen sulfide. Alternatively or additionally, the apparatus may be configured to detect methyl mercaptan, and dimethyl sulfide. In some variations the sensor may be configured to detect a salivary levels of an enzyme indicating the presence of certain halitosis-related bacteria, such as β-galactosidase.

Any of the apparatuses described herein may include microfluidics (e.g., lab-on-a-chip) components. Microfluidics may be used as part of the biosensor, for example, including channels for acquiring a biological fluid (e.g., saliva and/or GCF), processing the fluid (e.g., combining with one or more reagents and/or detecting an interaction with a biomolecule, etc.). The microneedles may be coupled to any of the microfluidics components.

Also included herein is the use of one or more biosensors (e.g., integrated with an orthodontic appliance such as an aligner) to detect either a protein or DNA component of an allergen. See, e.g., Alves et al., describing a biosensor system for food allergen detection (e.g., Alves et al. 2015. DOI:10.1080/10408398.2013.831026). As described herein, biosensors are well-suited to automation and their ability to detect multiple analytes in one test with minimal sample preparation and may be used to conduct in vivo detection and detect the presence of food allergens in close to real-time. For example, surface plasmon resonance (SPR)-based biosensors, typically used to the fast detection of egg-related fining allergens in wines, can be integrated with any of the orthodontic appliances (e.g., aligners) described herein to allow rapid detection the presence of egg white allergens at concentrations between 0.03 and 0.20 μg/mL.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

In general, the methods and apparatuses described herein may be used for monitoring the progress of appliance-based orthodontic treatment and/or compliance. Generally, a monitoring apparatus may include one or more biosensors and/or sensors (e.g., physiological sensors) configured to generate sensor data; this data may be related to repositioning of a patient's teeth using an orthodontic appliance. The biosensor and/or sensor data can be processed and analyzed to determine whether the appliance is successfully repositioning the teeth according to prescribed treatment plan. Advantageously, also described herein are integrated electronic sensing and logging systems capable of generating more reliable and accurate aligner performance data, which may be used by the treating practitioner to track treatment progress and adjust the patient's treatment plan if desired. The monitoring devices of the present disclosure can provide high value sensing data useful for adaptive closed-loop treatment planning and appliance design.

Monitoring performance of an orthodontic appliance for repositioning a patient's teeth is described. The apparatus can comprise an orthodontic appliance comprising one or more teeth-receiving cavities shaped to reposition the patient's teeth from an initial arrangement towards a target arrangement. The device can comprise one or more sensors configured to generate sensor data related to the repositioning of the patient's teeth by the orthodontic appliance. The device can comprise a processor configured to process the biosensor/sensor data in order to evaluate the performance of the orthodontic appliance in effecting the repositioning of the patient's teeth.

The performance of the orthodontic appliance can be measured in a variety of ways. For example, the processor may be configured to evaluate the performance of the orthodontic appliance by using the biosensor and/or sensor data to determine one or more of: an amount of force or pressure applied to the patient's teeth, a distribution of force or pressure on the patient's teeth, an amount of movement of the patient's teeth, or a movement rate of the patient's teeth, and/or the phase of movement of the patient's teeth (e.g., initial phase, a lag phase, and a post-lag phase, etc.).

The performance of the orthodontic appliance can determine one or more of: determining if movement is happening, what phase of tooth movement the patient is experiencing, and/or the rate of tooth movement. This information may be processed on the orthodontic appliance itself or off of the appliance (in a remote processor, etc.) and communicated to the dental professional and/or patient. This information may be used to adjust the treatment plan, including instruction removal of an orthodontic appliance (e.g., a removable orthodontic appliance/device) ahead of a scheduled removal, leaving the orthodontic appliance on longer than a scheduled removal date, or maintaining the scheduled removal/replacement date. In some variations the information (e.g., the information derived by monitoring the level of one or more biomarkers using the biosensors) may be used to modify the treatment plan by triggering replacement of one or more devices (aligners) within a planned sequence of removable orthodontic appliances.

In addition to the biosensors described herein, any of these methods and apparatuses may include a force or pressure sensor configured to measure force or pressure applied to one or more teeth by the orthodontic appliance. A force or pressure sensor can comprise a force- or pressure-sensitive film, a resistive film, a capacitive film, or a piezoelectric tactile sensor. The processor can be configured to evaluate the performance of the orthodontic appliance by determining whether an amount of force or pressure applied to the patient's teeth by the orthodontic appliance is within a targeted range. Any of these sensors may be used in combination with one or more biosensor, e.g., to confirm or estimate movement of the teeth.

A movement sensor may be included and configured to measure movement of one or more teeth. A movement sensor can comprise an electromagnetic field generator configured to generate an electromagnetic field. A movement sensor can be configured to measure the movement of the one or more teeth by measuring changes to the electromagnetic field. For instance, a movement sensor can comprise one or more electromagnetic targets arranged to move in response to the movement of the one or more teeth, such that movement of the one or more electromagnetic targets produces changes to the electromagnetic field.

Any of the apparatuses described herein may include a plurality of different biosensor and/or sensors operably coupled to different portions of the orthodontic appliance. The same or different microneedles may be coupled with one or more different biosensors. Any or all of these biosensors/sensors and microneedles can be integrated with the orthodontic appliance, coupled to a tooth, or a combination thereof. As discussed above, a processor may be integrated with the orthodontic appliance or coupled to a tooth. Alternatively, a processor can be located external to the patient's intraoral cavity. Any of these apparatuses my further comprises a communication module configured to transmit one or more of the sensor data or the processed sensor data to a remote device.

A method for monitoring performance of an orthodontic appliance for repositioning a patient's teeth may include receiving biosensor and/or sensor data related to the repositioning of the patient's teeth by the orthodontic appliance from one or more biosensors and/or sensors. The orthodontic appliance can comprise one or more teeth-receiving cavities shaped to reposition the patient's teeth from an initial arrangement towards a target arrangement. The biosensor data can be processed in order to evaluate the performance of the orthodontic appliance in effecting the repositioning of the patient's teeth.

Performance of the orthodontic appliance may be evaluated by using the biosensor data to determine one or more of: the state of a biomarker associated with tooth movement and/or remodeling, an amount of force or pressure applied to the patient's teeth, a distribution of force or pressure on the patient's teeth, an amount of movement of the patient's teeth, or a movement rate of the patient's teeth.

The apparatuses and methods described herein may include transmitting (e.g., wirelessly transmitting) one or more of the sensor and/or biosensor data or the processed sensor data to a remote device.

As mentioned above, the methods and apparatuses described herein can be used in combination with various types of orthodontic appliances. For example, appliances may have teeth-receiving cavities that receive and/or reposition teeth, e.g., via application of force due to appliance resiliency. The appliance can include a shell having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. In some embodiments, a physical appliance is directly fabricated, e.g., using rapid prototyping fabrication techniques, from a digital model of an appliance. Other orthodontic appliances may include the biosensors and microneedles described herein, including, e.g., palatal expanders. The methods and apparatuses described herein may be used with any appliance that receives teeth, for example appliances without one or more of polymers or shells. The appliance can be fabricated with one or more of many materials such as metal, glass, reinforced fibers, carbon fiber, composites, reinforced composites, aluminum, biological materials, and combinations thereof for example. The appliance can be shaped in many ways, such as with thermoforming or direct fabrication (e.g., 3D printing, additive manufacturing), for example. Alternatively or in combination, the appliance can be fabricated with machining such as an appliance fabricated from a block of material with computer numeric control machining.

An appliance can fit over all teeth present in an upper or lower jaw, or less than all of the teeth. The appliance can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some embodiments, some, most, or even all of the teeth will be repositioned at some point during treatment. Teeth that are moved can also serve as a base or anchor for holding the appliance as it is worn by the patient. Typically, no wires or other means will be provided for holding an appliance in place over the teeth. In some cases, however, it may be desirable or necessary to provide individual attachments or other anchoring elements on teeth with corresponding receptacles or apertures in the appliance so that the appliance can apply a selected force on the tooth.

As used herein, a dental appliance may include an aligner, such as those utilized in the Invisalign® System, which are described in numerous patents and patent applications assigned to Align Technology, Inc., including, for example, in U.S. Pat. Nos. 6,450,807, and 5,975,893, as well as on the company's website, which is accessible on the World Wide Web (see, e.g., the url “invisalign.com”). In this specification, the use of the terms “orthodontic aligner”, “aligner”, or “dental aligner” may be synonymous with the use of the terms “appliance” and “dental appliance” in terms of dental applications. For purposes of clarity, embodiments are hereinafter described within the context of the use and application of appliances, and more specifically “dental appliances.”

Examples of tooth-mounted attachments suitable for use with orthodontic appliances are also described in patents and patent applications assigned to Align Technology, Inc., including, for example, U.S. Pat. Nos. 6,309,215 and 6,830,450.

The orthodontic appliances described herein can be fabricated in a wide variety of ways. As an example, some embodiments of the appliances herein (or portions thereof) can be produced using indirect fabrication techniques, such as by thermoforming over a positive or negative mold. Indirect fabrication of an orthodontic appliance can involve producing a positive or negative mold of the patient's dentition in a target arrangement (e.g., by rapid prototyping, milling, etc.) and thermoforming one or more sheets of material over the mold in order to generate an appliance shell. Alternatively or in combination, some embodiments of the appliances herein may be directly fabricated, e.g., using rapid prototyping, stereolithography, 3D printing, and the like.

The configuration of the orthodontic appliances herein can be determined according to a treatment plan for a patient, e.g., a treatment plan involving successive administration of a plurality of appliances for incrementally repositioning teeth. Computer-based treatment planning and/or appliance manufacturing methods can be used in order to facilitate the design and fabrication of appliances. For instance, one or more of the appliance components described herein can be digitally designed and fabricated with the aid of computer-controlled manufacturing devices (e.g., computer numerical control (CNC) milling, computer-controlled rapid prototyping such as 3D printing, etc.). The computer-based methods presented herein can improve the accuracy, flexibility, and convenience of appliance fabrication.

Orthodontic appliances, such as the appliance illustrated in FIG. 1A, may impart forces to the crown of a tooth and/or an attachment positioned on the tooth at one or more points of contact between a tooth receiving cavity of the appliance and received tooth and/or attachment. The magnitude of each of these forces and/or their distribution on the surface of the tooth can determine the type of orthodontic tooth movement which results. Tooth movements may be in any direction in any plane of space, and may comprise one or more of rotation or translation along one or more axes. Types of tooth movements include extrusion, intrusion, rotation, tipping, translation, and root movement, and combinations thereof, as discussed further herein. Tooth movement of the crown greater than the movement of the root can be referred to as tipping. Equivalent movement of the crown and root can be referred to as translation. Movement of the root greater than the crown can be referred to as root movement.

The monitoring devices, including any of the microneedles, described herein can be physically integrated into an orthodontic appliance in a variety of ways. In some embodiments, the monitoring device is integrated into the appliance during or after fabrication of the appliance. For example, the monitoring device can be attached to an appliance using adhesives, fasteners, a latching mechanism, or a combination thereof after the appliance has been fabricated. Optionally, the appliance can be formed with complementary features or structures (e.g., recesses, receptacles, guides, apertures, etc.) shaped to receive and accommodate the monitoring device or components thereof.

A monitoring device may be coupled to the appliance as a prefabricated unit during or after fabrication of the appliance, such as by being inserted and sealed into a receptacle in the appliance, attached to an appliance (e.g., by a latching mechanism, adhesive, fastener). Alternatively, the monitoring device can be assembled in situ on the appliance during or after appliance fabrication. For instance, in embodiments where the appliance is manufactured by direct fabrication (e.g., 3D printing), the monitoring device can be printed simultaneously with the appliance, inserted into the appliance during fabrication, or after assembled the appliance has been fabricated. Optionally, some of the monitoring device components may be prefabricated and other components may be assembled in situ. It shall be appreciated that the various fabrication methods described herein can be combined in various ways in order to produce an appliance with integrated monitoring device components.

An orthodontic appliance can be operably coupled to a monitoring device configured to provide data related to tooth repositioning and/or the interaction between the appliance and the patient's teeth (e.g., contact between the appliance and the teeth, the amount of force and/or pressure applied by the appliance to the teeth, distribution of force and/or pressure on the teeth, etc.). Such data can be used to evaluate the performance of the orthodontic appliance for repositioning the patient's teeth. For instance, appliance performance information as described herein can include information regarding whether the force(s), pressure(s), and/or tooth movement(s) produced by an orthodontic appliance correlate with the expected values for the planned orthodontic treatment.

The monitoring devices described herein can be designed for use in the patient's intraoral cavity. For example, the dimensions of a monitoring device may be limited in order to avoid patient discomfort and/or facilitate integration into an orthodontic appliance as discussed below. In some embodiments, a monitoring device has a height or thickness less than or equal to about 1.5 mm, or less than or equal to about 2 mm. In some embodiments, a monitoring device has a length or width less than or equal to about 4 mm, or less than or equal to about 5 mm. The shape of the monitoring device can be varied as desired, e.g., circular, ellipsoidal, triangular, square, rectangular, etc. For instance, in some embodiments, a monitoring device can have a circular shape with a diameter less than or equal to about 5 mm.

A relatively thin and flexible monitoring device can be used to provide a larger surface area while reducing patient discomfort. In some embodiments, the monitoring devices herein are sized to conform to a surface of a tooth crown (e.g., a buccal, lingual, and/or occlusal surface of a tooth crown). For example, a monitoring device having dimensions of about 10 mm by about 5 mm can be used to cover a buccal surface of a molar crown. As another example, a monitoring device having dimensions of about 10 mm by about 20 mm can be used to cover the buccal, occlusal, and lingual surfaces of a tooth crown. A monitoring device can be in contact with a crown of a single tooth, or with crowns of a plurality of teeth, as desired.

The monitoring device dimensions (e.g., volume, weight) can be designed in order to reduce patient discomfort. For instance, the weight of a monitoring device can be selected not to exceed a level that would exert undesirable forces on the underlying teeth. A monitoring device may be used primarily for research and characterization purposes, rather than for patient treatment, and thus may not be subject to size constraints for reducing patient discomfort. For example, in embodiments where the monitoring device is used outside the intraoral cavity (e.g., benchtop testing of aligner performance), the size of the monitoring device can be relatively large compared to devices designed for intraoral use.

As discussed above, FIG. 7A schematically illustrates an example of an apparatus (e.g., a monitoring device 700 portion of an apparatus), that may be used or include an orthodontic appliance (not shown). The monitoring device 700 can be used in combination with any embodiment of the systems and devices described herein, including in combination with any of the microneedles describe herein, and the components of the monitoring device 700 are equally applicable to any other embodiment of the apparatuses described herein. The monitoring device 700 can be implemented as an application-specific integrated circuit (ASIC) including one or more of the following components: a processor 702, a memory 704, one or more biosensors and/or sensors 706, a clock 708, a communication unit 710, an antenna 712, a power management unit 714, or a power source 716. The processor 702 (e.g., a central processing unit (CPU), microprocessor, field programmable gate array (FPGA), logic or state machine circuit, etc.), also referred to herein as a controller, can be configured to perform the various methods described herein. The memory 704 encompasses various types of memory known to those of skill in the art, such as RAM (e.g., SRAM, DRAM), ROM (EPROM, PROM, MROM), or hybrid memory (e.g., flash, NVRAM, EEPROM), and the like. The memory 704 can be used to store instructions executable by the processor 702 to perform the methods provided herein. Additionally, the memory can be used to store biosensor/sensor data obtained by the biosensor(s)/sensors 706, as discussed in greater detail below. Either the biosensor 733 or the electronics subsystem 735 or both may include and/or be coupled with one or more microneedles 727 (in some variations including a bias 728) that may provide sample (e.g., saliva, blood, GCF, etc.) to the sensor(s).

The monitoring device 700 can include any number of biosensors 706 and/or sensor 706′, such as one, two, three, four, five, or more biosensors. In some embodiments, the use of multiple biosensors provides redundancy to increase the accuracy and reliability of the resultant data. Some or all of the biosensors 706 can be of the same type. Some or all of the biosensors 706 can be of different types. Examples of biosensor types suitable for use in the monitoring devices described herein are provided below. Examples of additional sensors may include: touch or tactile sensors (e.g., capacitive, resistive), proximity sensors, movement sensors (e.g., electromagnetic field sensors), force sensors (e.g., force-sensitive resistive or capacitive materials), pressure sensors (e.g., pressure-sensitive resistive or capacitive materials), strain gauges (e.g., resistive- or MEMS-based), electrical sensors, or combinations thereof.

A biosensor 706 can be operably coupled to and/or located at any portion of an orthodontic appliance, such as at or near a distal portion, a mesial portion, a buccal portion, a lingual portion, a gingival portion, an occlusal portion, or a combination thereof. A biosensor 706 can be positioned near a tissue of interest when the appliance is worn in the patient's mouth, such as near or adjacent the teeth, gingiva, palate, lips, tongue, checks, airway, or a combination thereof. For example, when the appliance is worn, the biosensor(s) 706 can cover a single tooth, or a portion of a single tooth. Alternatively, the biosensor(s) 706 can cover multiple teeth or portions thereof. In embodiments where multiple biosensors 706 are used, some or all of the monitoring devices can be located at different portions of the appliance and/or intraoral cavity. Alternatively, some or all of the biosensors 706 can be located at the same portion of the appliance and/or intraoral cavity.

An analog-to-digital converter (ADC) (not shown) can be used to convert analog biosensor and/or sensor data into digital format, if desired. The processor 702 can process the data obtained by the biosensor(s) 706 in order to determine appliance usage and/or patient compliance, as described herein. The biosensor data and/or processing results can be stored in the memory 704. Optionally, the stored data can be associated with a timestamp generated by the clock 708 (e.g., a real-time clock or counter).

In some embodiments, the monitoring device 700 incudes a communication unit 710 configured to transmit the data stored in the memory (e.g., biosensor data and/or processing results) to a remote device. The communication unit 710 can utilize any suitable communication method, such as wired or wireless communication methods (e.g., RFID, near-field communication, Bluetooth, ZigBee, infrared, etc.). The communication unit 710 can include a transmitter for transmitting data to the remote device and an antenna 712. Optionally, the communication unit 710 includes a receiver for receiving data from the remote device. In some embodiments, the communication channel utilized by the communication unit 710 can also be used to power the device 700, e.g., during data transfer or if the device 700 is used passively.

The remote device can be any computing device or system, such as a mobile device (e.g., smartphone), personal computer, laptop, tablet, wearable device, etc. Optionally, the remote device can be a part of or connected to a cloud computing system (“in the cloud”). The remote device can be associated with the patient, the treating practitioner, medical practitioners, researchers, etc. In some embodiments, the remote device is configured to process and analyze the data from the monitoring device 700, e.g., in order to assess appliance performance, for research purposes, and the like.

The monitoring device 700 can be powered by a power source 716, such as a battery. In some embodiments, the power source 716 is a printed and/or flexible battery, such as a zinc-carbon flexible battery, a zinc-manganese dioxide printed flexible battery, or a solid-state thin film lithium phosphorus oxynitride battery. The use of printed and/or flexible batteries can be advantageous for reducing the overall size of the monitoring device 700 and avoiding patient discomfort. For example, printed batteries can be fabricated in a wide variety of shapes and can be stacked to make three-dimensional structures, e.g., to conform the appliance and/or teeth geometries. Likewise, flexible batteries can be shaped to lie flush with the surfaces of the appliance and/or teeth. Alternatively or in combination, other types of batteries can be used, such as supercapacitors. In some embodiments, the power source 716 can utilize lower power energy harvesting methods (e.g., thermodynamic, electrodynamic, piezoelectric) in order to generate power for the monitoring device 700. Optionally, the power source 716 can be rechargeable, for example, using via inductive or wireless methods. In some embodiments, the patient can recharge the power source 716 when the appliance is not use. For example, the patient can remove the orthodontic appliance when brushing the teeth and place the appliance on an inductive power hub to recharge the power source 716.

Optionally, the apparatus can include a power management unit 714 connected to the power source 716. The power management unit 714 can be configured to control when the apparatus is active (e.g., using power from the power source 716) and when the apparatus inactive (e.g., not using power from the power source 716). In some embodiments, the monitoring device 700 is only active during certain times so as to lower power consumption and reduce the size of the power source 716, thus allowing for a smaller monitoring device 700

The apparatus may also include an activation mechanism (not shown) for controlling when the monitoring device (e.g., control circuitry) 700 is active (e.g., powered on, monitoring appliance usage) and when the monitoring device 700 is dormant (e.g., powered off, not monitoring appliance usage). The activation mechanism may deploy and/or retract the microneedles. The activation mechanism can be provided as a discrete component of the monitoring device 700, or can be implemented by the processor 702, the power management unit 714, or a combination thereof. The activation mechanism can be used to reduce the amount of power used by the monitoring device 700, e.g., by inactivating the device 700 when not in use, which can be beneficial for reducing the size of the power supply 716 and thus the overall device size.

In some embodiments, the monitoring device 700 is dormant before being delivered to the patient (e.g., during storage, shipment, etc.) and is activated only when ready for use. This approach can be beneficial in conserving power expenditure. For example, the components of the monitoring device 700 can be electrically coupled to the power source 716 at assembly, but may be in a dormant state until activated, e.g., by an external device such as a mobile device, personal computer, laptop, tablet, wearable device, power hub etc. The microneedles may be retracted (and in some variations may be housed within a housing). The external device can transmit a signal to the monitoring device 700 that causes the activation mechanism to activate the monitoring device 700, and extension of the microneedle(s). As another example, the activation mechanism can include a switch (e.g., mechanical, electronic, optical, magnetic, etc.), such that the power source 716 is not electrically coupled to the other components of the monitoring device 700 until the switch is triggered. For example, the switch may be a reed switch or other magnetic sensor that is held open by a magnet. The magnet can be removably attached to the monitoring device 700, or may be integrated into the packaging for the device 700 or appliance, for example. When the monitoring device is separated from the magnet (e.g., by removing the magnet or removing the device and appliance from the packaging), the switch closes and connects the power source 716, as illustrate in FIG. 7B. As another example, the monitoring device 700 can include a mechanical switch such as a push button that is manually actuated in order to connect the power source 716. In some embodiments, the activation mechanism includes a latching function that locks the switch upon the first actuation to maintain connectivity with the power source so as to maintain activation of the monitoring device 700. Optionally, the switch for the activation mechanism can be activated by a component in the patient's intraoral cavity (e.g., a magnet coupled to a patient's tooth), such that the monitoring device 700 is active only when the appliance is worn by the patient, and is inactive when the appliance is removed from the patient's mouth. Alternatively or in combination, the switch can be activated by other types of signals, such as an optical signal.

FIG. 7B illustrates a monitoring device 700 with an activation mechanism. The monitoring device 700, as with all other monitoring devices described herein, can be similar to the monitoring device 700, and can include some or all of the components described herein with respect to the monitoring device 700. The device 700 is coupled to an orthodontic appliance 702 (e.g., via an encapsulating material 404). The device 400 can include an activation mechanism 703 including a magnetic switch. Prior to use, the device 700 can be removably coupled to a magnet 706 (e.g., using tape 708), and the magnet 706 can hold the magnetic switch in an open position such that the device 700 is inactive. When the appliance 702 is ready for use, the user can remove the magnet 706, thus closing the magnetic switch and connecting the components of the monitoring device 700 to a power source.

The orthodontic appliances and monitoring devices can be configured in many different ways. In some embodiments, an orthodontic appliance may be operably coupled to a single monitoring device. Alternatively, the orthodontic appliance can be operably coupled to a plurality of monitoring devices, such as at least two, three, four, five, or more monitoring devices. Some or all of the monitoring devices may be of the same type (e.g., collect the same type of data). Alternatively, some or all of the monitoring devices may be of different types (e.g., collect different types of data). Any of the embodiments of monitoring devices described herein can be used in combination with other embodiments in a single orthodontic appliance.

A monitoring device, including one or more microneedles, can be located at any portion of the appliance, such as at or near a distal portion, a mesial portion, a buccal portion, a lingual portion, a gingival portion, an occlusal portion, or a combination thereof. The monitoring device can be positioned near a tissue of interest when the appliance is worn in the patient's mouth, such as near or adjacent the teeth, gingiva, palate, lips, tongue, checks, airway, or a combination thereof. For example, when the appliance is worn, the monitoring device can cover a single tooth, or a portion of a single tooth. Alternatively, the monitoring device can cover multiple teeth or portions thereof. In embodiments where multiple monitoring devices are used, some or all of the monitoring devices can be located at different portions of the appliance. Alternatively, some or all of the monitoring devices can be located at the same portion of the appliance.

A monitoring device can be operably coupled to the orthodontic appliance in a variety of ways. For example, the monitoring device can be physically integrated with the orthodontic appliance by coupling the monitoring device to a portion of the appliance (e.g., using adhesives, fasteners, latching, laminating, molding, etc.). The coupling may be a releasable coupling allowing for removal of the monitoring device from the appliance, or may be a permanent coupling in which the monitoring device is permanently affixed to the appliance. Alternatively or in combination, the monitoring device can be physically integrated with the orthodontic appliance by encapsulating, embedding, printing, or otherwise forming the monitoring device with the appliance. In some embodiments, the appliance includes a shell shaped to receive the patient's teeth, and the monitoring device is physically integrated with the shell. The monitoring device can be located on an inner surface of the shell (e.g., the surface adjacent to the received teeth), an outer surface of the shell (e.g., the surface away from the received teeth), or within a wall of the shell. Optionally, as discussed further herein, the shell can include a receptacle shaped to receive the monitoring device. Exemplary methods for fabricating an appliance with a physically integrated monitoring device (e.g., by incorporating some or all of the components of the monitoring device during direct fabrication of the appliance) are described in further detail herein.

FIG. 9 illustrate an example of an apparatus including an orthodontic appliance 900 having an integrated monitoring device (control circuitry) 902 and biosensor and one or more microneedles 917. In this example, the appliance 900 includes a shell 904 having one or more (e.g., a plurality of) teeth-receiving cavities, and the monitoring device 902 is coupled to an outer, buccal surface of the shell 904 adjacent a tooth receiving cavity 906. In the depicted embodiment, the monitoring device 902 is coupled to a tooth receiving cavity 906 for a molar. It shall be appreciated that in alternative embodiments, the monitoring device 902 can be coupled to other portions of the shell 904, such as an inner surface, a lingual surface, an occlusal surface, one or more tooth receiving cavities for other types of teeth (e.g., incisor, canine, premolar), etc. The monitoring device 902 can be shaped to conform to the geometry of the corresponding appliance portion (e.g., the wall of the cavity 906) so as to provide a lower surface profile and reduce patient discomfort. In some embodiments, the appliance 900 includes a receptacle 908 formed on the outer surface of the shell 904 and the monitoring device 902 is positioned within the receptacle. Exemplary methods for forming an appliance with a receptacle 908 and integrated monitoring device 902 are described in detail below.

The monitoring device 902 can include a biosensor 910 and/or sensor, one or more microneedles 917, a power source 912 (e.g., a battery), and/or a communication unit 914 (e.g., a wireless antenna). The arrangement of the components of the monitoring device 902 can be varied as desired. In some embodiments, the biosensor is located adjacent to the tooth receiving cavity. A gap can be formed in the shell adjacent to the biosensor/sensor so as to permit direct access to the received tooth. The communication unit (or a component thereof, such as an antenna) can be located adjacent to or on the outer surface of the receptacle so as to facilitate data transmission.

A monitoring device can include a single biosensor, or a plurality of biosensors and/or other sensors can be positioned at any location in the appliance, such on an inner surface, an outer surface, a buccal surface, a lingual surface, an occlusal surface, a mesial portion, a distal portion, a gingival portion, or a combination thereof. In embodiments where the orthodontic appliance includes a shell with a teeth-receiving cavity, the biosensors/sensors can be positioned on the inner surfaces of the teeth-receiving cavities. Optionally, at least some biosensors can be located on an outer surface of the appliance, such as an occlusal surface in order to detect contact between the upper and lower teeth

The biosensors can be positioned to be near certain teeth when the appliance is worn, e.g., near teeth to be repositioned and/or at locations where the appliance is expected to exert force on the teeth. For example, tactile sensors can be located at or near the buccal, lingual, and/or occlusal surfaces of a tooth to be repositioned so as to provide a map of contact points over the tooth crown. In some embodiments, the monitoring device is configured to obtain data from buccal, lingual, and occlusal sensors in a predetermined order and at a desired frequency in order to provide a contact map over the buccal, lingual, and occlusal surfaces. Alternatively or in combination, if the appliance is shaped to engage an attachment device mounted on a tooth, a tactile sensor can be located at or near the location of engagement between the appliance and the attachment device.

Alternatively or in combination, any of the apparatuses described herein can include one or more conductivity sensors configured to measure the conductivity of fluids (e.g., saliva) in the surrounding environment. In some embodiments, bone remodeling during orthodontic tooth movement causes changes in saliva content, and these changes can be measured based on the ionic charge of the minerals in the saliva. Examples of minerals that may influence the conductivity of saliva include but are not limited to NH4+, Ca2+, P043″, HC03−, and F″.

In general, the apparatuses described herein may include miniaturized and integrated electronic components (e.g., battery, antenna, controller, wireless communication circuitry, etc.) as part of an embedded biosensing apparatus. In some variations, the biosensor may include detection of one or more types of biomarker in saliva, including those in Table 1, above. For example, a Potentiostat with a screen-printed electrode and a modified enzyme layer may be used to detect a salivary biomarker (e.g., 14-3-3 protein σ (Stratifin), uric acid, etc.). A working electrode (biotransducer) may be chemically modified by crosslinking to an enzyme. An antifouling layer may be included to prevent interference effects and biofouling.

The apparatus may include electrodes for differential C2D (e.g., common to differential) measurements and input channels for A2D (analog to digital) voltage measurements. One or more electrodes may be layered with an enzyme-membrane to achieve different configurations of working electrodes. Additional sensors and/or biosensors may be used, including temperature measurements. For example, one or more sensors can provide proximity data, which can augment biomarker detection.

In any of these apparatuses and methods described herein, compliance data may be determined from the biosensor/sensor data, and this information may also be used to augment, control, and/or interpret the biosensor information. For example, compliance data may be estimated by determining a working state of the apparatus from proximity data (e.g., power states for “In-mouth” and “out-of-mouth” conditions). Temperature sensing may also be used to augment biomarker data, e.g., by correlating temperature and biomarker data, which may provide more specific physiological monitoring.

As discussed above, examples of biomarkers that may be detected by the apparatuses and methods herein may include salivary biomarkers such as sRANKL, OPG, which may be correlated to different phases of orthodontic tooth movement (e.g., https://www.ncbi.nlm.nih.gov/pubmed/23273364). Other salivary markers include S100-A9, immunoglobulin J chain, Ig alpha-1 chain C region, CRISP-3, which may indicate inflammation and bone resorption (sec, e.g., https://www.ncbi.nlm.nih.gov/pmc/articles/PMC3417200/). Examples of gingival crevicular fluid biomarkers, e.g., inflammatory fluid accessible in in the gingival margin, may include prostaglandin E2 (which may indicate bone resorption), Substance P (neuropeptide) (which may indicate bone resorption), epidermal growth factor (which may indicate bone resorption), transforming growth factor (which may indicate bone remodeling), RankL (which may indicate stimulation of osteoclsatic differentiation), Granulocyet macrophage colony stimulation factor (which may indicate bone turnover), α2 microglobulin-enhance of IGF 1, Interleukin 1β, 2, 6, 8 (which may indicate bone remodeling), Myeloperoxidase (and enzyme involved in PMN inflammation). Other gingival crevicular fluid biomarkers may include glycosaminoglycans (GAGs or mucopolysaccharides), and may indicate paradental remodeling, such as hyaluronic acid (a type of GAG or mucopolysaccharide, indicator of breakdown of gingival tissue), and Chondroitin sulfate (another type of GAG, an indicator of breakdown of alveolar bone and PDL).

Alternatively, as mentioned, a sensor, such as a stretchable sensor, may be bonded directly to the teeth or other intra oral tissue. This may provide better access to GCF or saliva for the biosensor/sensor. Instead of an embedded biosensor on the aligner, the biosensor may be directly bonded to the gingival margin to monitor GCF for certain biomarkers.

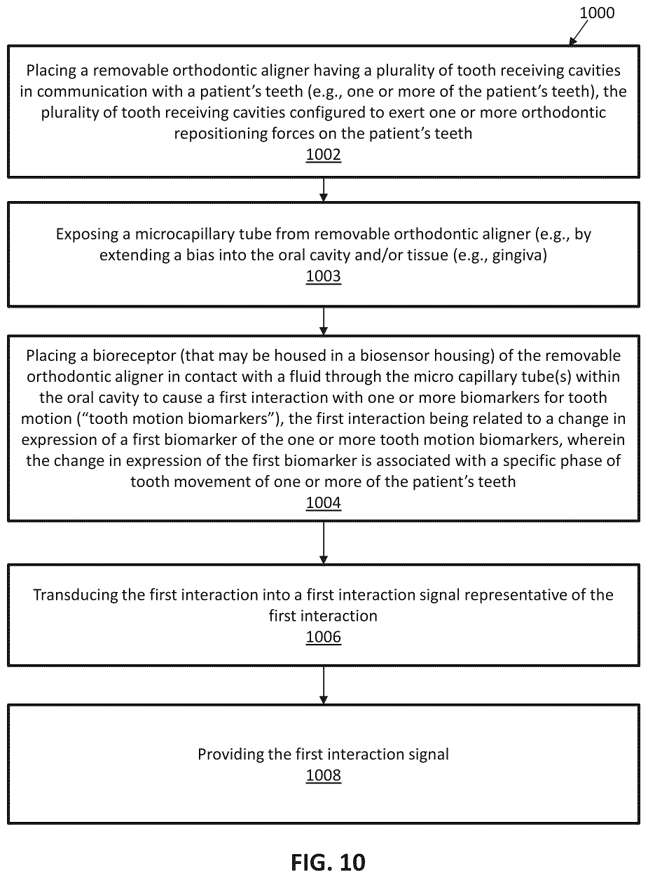

As mentioned above, any of the biosensor systems and apparatuses (e.g., removable orthodontic devices) described herein may be used to monitor one or more biomarkers from a patient. For example, FIG. 10 schematically illustrates one example of a method of monitoring a biomarker from a patient using a removable orthodontic device, such as an aligner, that includes one or more microneedles for detecting a biomarker. As mentioned above, as an initial step (not shown) a removable orthodontic device that does not apply substantial force to move the teeth but that includes a biosensor system may be first worn, and a baseline for the one or more biomarkers collected. In FIG. 10, a removable orthodontic device (e.g., aligner) having a biosensor may be worn by the patient. For example, the removable orthodontic device may have a plurality of tooth receiving cavities that may be placed in communication with a patient's teeth (e.g., one or more of the patient's teeth). The plurality of tooth receiving cavities may be configured to exert one or more orthodontic repositioning forces on the patient's teeth 1002.

While the aligner is worn, the bioreceptor (that may be housed in a biosensor housing) of the removable orthodontic aligner may be placed in contact with a fluid (e.g., saliva, GCF, blood) within the oral cavity, e.g., through the microneedle(s) 1003, as described above, which may cause a first interaction with one or more biomarkers for tooth motion (“tooth motion biomarkers”). The first interaction may be related to a change in expression of a first biomarker of the one or more tooth motion biomarkers, wherein the change in expression of the first biomarker is associated with a specific phase of tooth movement of one or more of the patient's teeth 1004. For example, the level of the biomarker compared to a baseline may be indicative of the phase of tooth movement (e.g., initial phase, lag phase, etc.). A threshold or range of sensed values may be used to monitor an effect of the removable orthodontic device on the teeth. For example, the first interaction may be transduced into a first interaction signal representative of the first interaction 1006, and this first interaction signal may be provided by the device 1008. For example it may be output (transmitted, displayed, stored, etc.).

As mentioned above, any of the biosensors described herein may include one or more microfluidics systems for capture, storage and analysis of intra-oral fluids, including chemical analysis. For example, any of these microfluidic systems may include hard or flexible/stretchable (such as silicone) materials with or without integrated electronics (including wireless communication electronics), and may be formed integrally with the apparatus (e.g., aligner) and/or be intimately and robustly bond to the surface of apparatus. The one or more microneedles described herein may be fluidically linked to the microfluidics system(s).

In some variations, the microfluidic system may include a network of reservoirs for embedded chemical agents that may respond in colorimetric fashion to biomarkers. The reservoirs may be connected by microfluidics channels. In some variations the microfluidics channels may be configured for active and/or passive metering, so that a fluid from within the patient's oral cavity (e.g., saliva and/or GCF) may be drawn into the microfluidics channel (e.g., from the microneedle(s) as described herein) and passed into a sample chamber. The sample chamber may include, for example a colorometric indicator or other chemical agent that responds to one or more biomarkers in the fluid in a colorimetric manner. Alternatively, in some variations, the sample is processed in a microfluidics channel for later read-out (e.g., when removing the device from the mouth, and placing it into a separate storage and/or readout chamber.

In any of these variations, apparatus may include microfluidic channels that are configured to allow access to various sample and/or detection regions on the apparatus at various times. For example, the microfluidics device integrated into or on an aligner may be configured to provide timing via chrono-sampling of a fluid. For example, a microfluidic system can be designed to enable sampling with chronological order and controlled timing. In some variations, the timing of fluid within the microchannel may be timed actively, e.g., by the opening of a channel via release of a valve (e.g., an electromechanical valve, an electromagnetic value, a pressure valve, etc.). Examples of valves controlling fluid in a microfluidic network include piezoelectric, electrokinetics and chemical approaches. Capillary bursting valves (CBVs) are another variation of a valve for a microfluidics channel. CBVs block flows at pressures lower than their characteristic bursting pressures (BPs). When liquid in a single connected channel encounters two separate CBVs with different BPs, at sufficient pressures, the flow will proceed first through the valve with lower BP. In this way, locating two CBVs with different BPs near the intersection between two channels allows control of the direction of flow. The Young-Laplace equation gives the BP in a rectangular channel:

$$BP = - 2 σ [ \frac{cos \text{?}}{b} + \frac{cos \text{?}}{h} ] \qquad [ 1 ]$$

$$\text{?} \text{indicates text missing or illegible when filed}$$

where σ is the surface tension of liquid, θ_{A} is the contact angle of the channel, θ₁* is the min[θ_{A}+β; 180°], β is the diverging angle of the channel, b and h are the width and the height of the diverging section, respectively. For hydrophobic materials at high diverging angles, the BP increases with decreasing b and h.

Thus, by adjusting the angles and dimensions, the bursting pressure may be adjusted and in the context of a microfluidics channel, series of CBVs may be used to set up a sequence of timed regions that open as the fluid within the channel reaches the selected BP. For example, the diverging angles may be between 13° and 90°, or 13° and 120°. One of skill in the art would therefore be able to select the microfluidic channel lengths and BP configurations to arrange a series of microfluidics channels and chambers, which are valved by one or more control valves, including CBVs that open at predetermined time ranges to allow sampling over time.

In some variations the microfluidic channel may be opened by dissolving a material blocking the channel. The rate of dissolution may be calibrate to open the channel after a predetermined time period (e.g., minutes, hours, days, etc.). In some variations the dissolving material may include one or more reagents for use in detecting and/or storing the fluid in the microfluidics apparatus. For example, a blocking material may include a labeling material (e.g., such as an antibody, enzyme, substrate, etc.) for detection within a chamber blocked by the blocking material.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for case of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An intraoral system, comprising:
a polymeric shell appliance comprising a plurality of cavities for receiving a patient's teeth, wherein the polymeric shell appliance is configured to be removably worn over the patient's teeth; and
a sensor assembly coupled to the polymeric shell appliance, the sensor assembly comprising a power source, a sensor that is configured to detect air flow during inhalation and/or exhalation of the patient when the patient is wearing the polymeric shell appliance, a memory for storing air flow data based on the detected air flow, and wireless circuity for transmitting information associated with the air flow data to an external device.

2. The intraoral system of claim 1, wherein the sensor assembly comprises one or more processors configured to determine, based on the air flow data, a respiration rate of the patient.

3. The intraoral system of claim 1, wherein the sensor assembly comprises one or more processors configured to determine, based on the air flow data, whether the patient is snoring or has sleep apnea during sleep.

4. The intraoral system of claim 1, wherein the polymeric shell appliance is shaped to resiliently reposition the patient's teeth according to a phase of an orthodontic treatment plan.

5. The intraoral system of claim 1, wherein the sensor assembly is configured to activate the patient's muscles in response to determining snoring or sleep apnea to encourage or cause the patient's airways to open.

6. The intraoral system of claim 3, wherein the sensor assembly is configured to deliver electrical stimulation to the patient in response to detecting the snoring or sleep apnea to encourage or cause the patient's airways to open.

7. The intraoral system of claim 1, wherein the sensor assembly is configured to analyze air inhaled by the patient.

8. The intraoral system of claim 7, wherein the sensor assembly is configured to analyze toxic or harmful substances in the inhaled air and warn the patient of the toxic or harmful substances.

9. The intraoral system of claim 1, wherein the sensor assembly is configured to analyze air exhaled by the patient to detect volatile and/or non-volatile compounds.

10. The intraoral system of claim 1, wherein the sensor assembly is configured to analyze air exhaled by the patient to detect an alcohol level.

11. The intraoral system of claim 1, wherein the sensor assembly is configured to analyze air exhaled by the patient to detect one or more markers for bad breath.

12. The intraoral system of claim 1, wherein the sensor assembly includes microfluidic components associated with analyzing chemical content of air exhaled by the patient and/or air inhaled by the patient.

13. A method, comprising:
placing a polymeric shell appliance over a patient's teeth such that the patient's teeth are positioned within a plurality of cavities of the polymeric shell appliance, wherein the polymeric shell appliance is configured to be removable from the patient's teeth;
detecting air flow during inhalation and/or exhalation of a patient, wherein the air flow is detected by a sensor of a sensor assembly coupled with the polymeric shell appliance worn by the patient, wherein the sensor assembly is powered by a power source coupled with the polymeric shell appliance;
storing air flow data based on the detected air flow in a memory of the sensory assembly; and
transmitting information associated with the air flow data to an external device via wireless circuitry of the sensor assembly.

14. The method of claim 13, wherein the sensor assembly determines a respiration rate of the patient.

15. The method of claim 13, wherein the sensor assembly detects whether the patient is snoring or has sleep apnea during sleep.

16. The method of claim 15, further comprising delivering electrical stimulation to the patient in response to detecting the snoring or sleep apnea to encourage or cause the patient's airways to open.

17. The method of claim 13, wherein the sensor assembly analyzes air inhaled by the patient.

18. An intraoral system, comprising:
a polymeric shell appliance comprising a plurality of cavities for receiving a patient's teeth, wherein the polymeric shell appliance is configured to be removably worn over the patient's teeth; and
a sensor assembly coupled to the polymeric shell appliance, the sensor assembly comprising a power source, a sensor that is configured to detect air flow during inhalation and/or exhalation of the patient when the patient is wearing the polymeric shell appliance, a memory for storing air flow data based on the detected air flow, and wireless circuity for transmitting information associated with the air flow data to an external device, wherein the sensor assembly is configured to detect whether the patient is snoring or has sleep apnea during sleep.

19. The intraoral system of claim 18, wherein the polymeric shell appliance is shaped to resiliently reposition the patient's teeth according to a phase of an orthodontic treatment plan.

20. The intraoral system of claim 18, wherein the sensor assembly comprises one or more processors configured to determine, based on the air flow data, a respiration rate of the patient.
